(19) United States
(12) Patent Application Publication
(10) Pub. No.: US 2009/0221469 A1
(43) Pub. Date: Sep. 3, 2009

(54) USE OF SOY KEFIR POWDER FOR REDUCING PAIN, BLOOD PRESSURE AND INFLAMMATION

(75) Inventors: Stan Kubow, Pointe-Claire (CA); John Sheppard, Cary, NC (US)

Correspondence Address: DARBY & DARBY P.C., P.O. BOX 770, Church Street Station, New York, NY, 10008-0770, US

(73) Assignee: KCLM RESEARCH IN NUTRITION INC., Beaconfield, QC (CA)

(21) Appl. No.: 12/278,217

(22) PCT Filed: Feb. 2, 2007
(86) PCT No.: PCT/CA2007/000152

§ 371 Date: Feb. 3, 2009

Related U.S. Application Data
(60) Provisional application No. 60/764,372, filed on Feb. 2, 2006.

(51) Int. Cl. A61K 31/70; A61K 31/352; A61K 38/02; A61P 29/00; A61P 25/00

(52) U.S. Cl. 514/2; 514/456; 514/23

(57) ABSTRACT

The present invention relates to a soy kefir powder produced by the fermentation of soy milk with Kefir grains of the Moscow strain under suitable fermentation conditions. The soy kefir powder of the invention has at least one of the following biological activities: pain relief, blood pressure reduction and inflammation reduction. The present invention also relates to the method of production of the soy kefir powder of the invention. It also relates to the use of the soy kefir powder of the invention for pain relief, blood pressure reduction and inflammation reduction. The invention also relates to methods for pain relief, blood pressure reduction and inflammation reduction by using the soy kefir powder of the invention.

### FIELD OF THE INVENTION

The present invention relates to kefir, and more particularly to soy kefir powder and its use in pain relief, blood pressure reduction and/or inflammation reduction. The present invention also relates to the method of production of such soy kefir powder.

### BRIEF DESCRIPTION OF THE PRIOR ART

Kefir originates from the Northern Caucasus Mountains where it has been consumed for centuries and has been valued for numerous health promoting properties⁶. It continues to be a popular beverage in Eastern Europe, Scandinavia, and numerous individual countries^{13,14}. In the former Soviet Union, kefir has been traditionally used in hospitals and sanatoria for the treatment of numerous conditions including metabolic disorders, atherosclerosis, allergic disease, peptic ulcers, biliary tract diseases, chronic enteritis, bronchitis and pneumonia. It has also been used to treat tuberculosis, cancer, and gastrointestinal disorders when medical treatment was unavailable.⁶

Kefir grains are not to be mistaken for cereal grains, i.e., the grain part of the name is a misnomer. Kefir grains, or kefir granules are in fact a natural mother-culture. The grains are a soft, gelatinous white biomass, comprised of protein, lipids and a soluble-polysaccharide complex called kefiran.

Kefir grains are clusters of microorganisms held together by the Kefiran polysaccharides. Kefiran provides for a stable matrix that functions as a natural immobilized cell system. Kefiranofaciens and L. kefir produce these polysaccharides. The polysaccharides are an integral part of the grain, and without their presence, kefir grains cannot be propagated. The grains resemble small cauliflower florets. They are a soft white gelatinous mass. Each grain is 3 to 20 mm in diameter. Their structure being the result of a symbiotic relationship shared between a large variety of specific lactic acid bacteria and yeasts. The grain matrix is composed of a complex of 13% protein (by dry weight), 24% polysaccharide, plus cellular debris and unknown components²⁻¹². The kefir grains ferment the milk, incorporating their probiotic organisms to create the cultured product. Kefir is a cultured milk beverage made by adding kefir grains to various milk products (i.e., cow, goat, soy, and other commonly consumed milks).

Kefir grains are not consumed as part of the final product; they are removed with a strainer at the completion of fermentation and added to a new batch of unfermented milk. The grains contain a relatively stable and specific balance of microorganisms, which exist in a complex symbiotic relationship. The grains are formed in the process of making kefir and only from pre-existing grains. The grains include primarily lactic acid bacteria (lactobacilli, lactococci, leuconostocs) and yeast. Varieties of yeasts such as Kluyveromyces, Candida, Torulopsis, and Saccharomyces sp. are also present in kefir grains. Certain yeasts of kefir include the name Candida as part of their nomenclature. These kefir yeasts are not opportunistic yeasts such as C. albicans, but are classified as Generally Regarded As Safe (GRAS). Candida albicans has not been found in kefir grains. The dominant microflora are Saccharomyces kefir, Lactobacillus caucasicus, Leuconnostoc species and lactic streptococci. Other probiotic microorganisms present in the grains include lactobacilli, such as Lb. acidophilus, Lb. brevis, Lb. casei, Lb. casei subsp. rhamnosus, Lb. casei subsp. Pseudoplantarum, Lb. paracasei subsp. paracase, Lb. cellobiosus, Lb. delbrueckii subsp. bulgaricus, Lb. delbrueckii subsp. lactis, Lb. fructivoran, Lb. helveticus subsp. lactis, Lb. hilgardii, Lb. kefiri, Lb. kefiranofaciens, Lb. kefirgranum sp. nov, Lb. parakefir sp. nov, Lb. lactis, Lb. plantarum, Lb. cellobiosus, Lb. helveticus, Lactococci are also present such as subspecies of Lc. lactis, Lc. lactis var. diacetylactis, Lc. lactis subsp. Cremoris, Leuconostoc mesenteroides, Leuconostoc cremoris and L. cremoris, Streptococci salivarius subsp. thermophilus, and S. lactis, Enterococcus durans. Other bacteria include Acetobacter aceti and A. rasen.²⁻⁸ Such yeasts may have the potential to keep C. albicans under control in the host. The mean ranges of unit counts of microbes in gram stained kefir grains are, a) bacilli, 62-69%, b) streptococci 11-12%, and c) yeast, 16-20%.^{2,3,7,10-12}

The beverage kefir has a tart, refreshing taste that is slightly acidic due to the presence of lactic acid. It is naturally effervescence due to the presence of carbon dioxide and minute concentrations of alcohol (i.e., 0.08% to 2%) as a result of yeast fermentation. Kefir also contains a variety of approximately 40 aromatic compounds, including diacetyl and acetaldehyde, which give it a characteristic flavour and aroma.¹

Bacteriocin may also be present, especially if the appropriate strains of lactic acid bacteria are present in the grains.^{2,9}

As the microbial composition varies significantly according to the kefir grain source, the source is critical to determining the final composition of the kefir product.^{2,3} The wide variety of microorganisms used in kefir fermentation differentiates kefir from virtually all other cultured milk products, which typically use only one and rarely more than three species in the culturing process.

Extracts of fermented soy foods have angiotensin converting enzyme (ACE) inhibitory and blood pressure (BP) lowering properties comparable to those of ACE inhibitor drugs.³³ Soy hydrolysates and soy ACE inhibitory peptides have been demonstrated to inhibit ACE activity in vascular tissue and to lower systolic blood pressure (SBP) in spontaneously hypertensive rats.³³⁻³⁵ Moreover, anti-hypertensive effects have been obtained from milk fermented with a combination of various lactic acid bacteria and yeast, a process analogous to kefir fermentation, albeit that kefir grains contain a greater variety of bacteria and yeast.²⁷

ACE raises BP by converting angiotensin I (AI), released from angiotensinogen by renin, into the potent vasoconstrictor angiotensin II (AII). ACE also degrades vasodilative bradykinin in blood vessels and stimulates the release of aldosterone in the adrenal cortex. Therefore, agents that inhibit ACE, and subsequently reduce circulating and local levels of AII, are effective modalities for the treatment of hypertension.³⁶

Furthermore, AII has significant proinflammatory actions in the vascular wall, inducing the production of oxidative stress, inflammatory cytokines, and adhesion molecules.³⁷ AII induces the synthesis and secretion of IL-6, a cytokine that induces synthesis of angiotensinogen and subsequent BP elevation.³⁸ IL-6 also plays an important role in upregulating C-reactive protein (CRP),³⁹ which is also involved in the development of hypertension.⁴⁰ Conversely, CRP declines with ACE inhibitor treatment.⁴¹ In addition to being implicated in the development of hypertension, baseline levels of CRP and IL-6 are independently associated with increased risk of developing heart disease.⁴²

Other putative bioactive ingredients in soy kefir are isoflavones. Soybeans contain the highest natural concentration of isoflavones of any food.⁴³ The major dietary isoflavones found in soy are genistein, daidzein, formononetin, biochanin A and coumestrol. The biologically active isoflavones, genistein and daidzein, are substantially increased with soy protein fermentation.⁴⁴

Soy isoflavones have been shown to possess anti-hypertensive and anti-inflammatory properties. For example, genistein has shown potent anti-hypertensive effects in spontaneously hypertensive rats.⁴⁵ Isoflavones also inhibit the co-transport of sodium, potassium, and chloride, mimicking the actions of loop diuretics.⁴⁶ In addition to natriuresis, genistein and equol exert vasorelaxation in animal models.⁴⁷⁻⁹ Furthermore, quercetin, a flavonoid analog of genistein, may exert antihypertensive effects via its antioxidant capabilities.⁵⁰

Fermentation of food proteins increases their digestibility and allows for greater absorption of peptides, without changing the overall biological value.⁷⁰ In particular, proteins with high disulfide content such as soy are relatively resistant to digestion,⁷¹ and fermentation increases their digestibility to allow for greater absorption of peptides.⁷⁰⁻⁷² Some physiologically active bioactive peptides may be present in their inactive forms in the amino acid sequences of proteins and are normally poorly absorbed from undigested soy proteins.

Fermentation may release these “hidden” peptides and subsequently exert health benefits. Small dipeptides and tripeptides, and even large peptides (10-51 amino acids) can be absorbed intact through the intestines and produce biological effects.^{73,74} It is noteworthy that ACE inhibitory peptides derived from milk fermentation have been shown to be resistant to the digestive condition and to exert a BP lowering effects when given orally to spontaneously hypertensive rats.⁷⁵ Isoflavonoids undergo acidic and enzymatic hydrolysis in the human gut and the isoflavones, biochanin A and formononetin, undergo demethylation to yield the aglycones genistein and daidzein, respectively. This metabolism may vary among individuals, resulting in differences in the relative proportions of isoflavonoid metabolites produced in the gut.⁷⁶

The half-lives of isoflavones are about 4-8 h, which suggests that maintenance of high plasma concentrations of isoflavone metabolites could be achieved with regular and frequent consumption of soy products.⁷⁷

For centuries, Asians have consumed fermented soy products with ACE inhibitory activity such as soy sauce and natto,^{78,79} with no documented adverse effects being noted apart from an adverse drug-food interaction noted with monoamine oxidase inhibitor drugs.^{80,81} While the presence of isoflavones with putative hormonal like activities (i.e., genistein and daidzein) may cause some safety concern, a review of the literature indicates that 40 g of soy powder contains 6-23.2 mg daidzein and 0.076-33.6 mg genistein. A typical 60 kg person consuming 40 g soy powder/day will not be exposed to more than 0.39 mg/kg/day daidzein or 0.56 mg/kg/day genistein. Animal studies, while limited, demonstrate that adverse effects were only observed at levels of isoflavones that are at least approximately 100 times higher than that found in 40 g of soy powder (see Example 2).

Recently, it has been confirmed that highly concentrated, filtered extracts derived from soymilk fermented with bacteria and/or yeasts have been provided to human subjects (i.e., infants, asthmatic children, pregnant and lactating mothers, women undergoing surgery) with no noted adverse effects.^{20,21} These same extracts have undergone acute and chronic toxicity studies in rodents showing no signs of toxicity.^{20,21} They are non-mutagenic in Ames test, they do not cause in vitro mammalian cell chromosomal damage, nor do they induce micronuclei in bone marrow cells in ICR mice.^{20,21} While single doses (5 mL/kg) of fermented milk products have led to Systolic Blood Pressure (SBP) reductions in hypertensive rats, no reductions were noted in normal rats.⁸² Indeed, oral administration of soy ACE inhibitory peptides (100, 500, and 1000 mg/kg/day) demonstrated no BP reduction in normotensive rats even at the highest doses, whereas a linear dose trend was observed in spontaneously hypertensive rats.^{33,34} Other animal and human studies of fermented milk and protein hydrolysates have consistently demonstrated an absence of blood pressuring effects in both normotensive rats and humans.⁸⁰

There is known in the art controlled clinical trials that have investigated some product's efficacy and safety in the treatment of hypertension

For instance, in a 3-month double-blind study of men and women with mild-to-moderate hypertension, the antihypertensive potential of unfermented soymilk compared with unfermented cow's milk was investigated.⁹² After unfermented soymilk consumption, SBP decreased compared to the cow's milk group, and DBP decreased compared to the cow's milk group.

The hypotensive action of chronic soymilk consumption was correlated with the urinary excretion of the isoflavonoid genistein. There were no reports of adverse events for either treatment group.

In another study, hypertensive patients received either a test product (L. helveticus LBK-16H fermented cow's milk) or a control product (Lactococcus sp. fermented cow's milk)⁹³. Compared to the BP reductions noted with the control product, the test product induced greater reduction in SBP.

A further placebo controlled study of mildly hypertensive patients was conducted using FMG, a GABA containing fermented milk product.⁹⁵ A significant decrease of BP was noted within 2 to 4 weeks; an effect that was maintained throughout the 12-week dosing period. Furthermore, SBP reduction in the FMG group was significantly greater than the reduction obtained with placebo. There were no notable adverse events, and heart rate, body weight, haematology, blood chemistry and urinalysis results were similar between treatment groups.

Although some clinical studies seem to indicate that soy and/or soy isoflavones have the capacity of lowering blood pressure in hypertensive subjects, there are also clinical evidences on soy that does not support such hypothesis since no significant decrease of BP was observed⁹⁹⁻¹⁰⁹ (see also Table 1). Furthermore, this fact is also the conclusion of a major review on the cardiovascular effects of soy proteins.¹¹⁰

Pain relief from neuropathic pain from intake of soy protein has been implicated in rat studies (Shir Y, Sheth R, Campbell J N, Raja S N, Seltzer Z. Anesth Analg. 2001 April; 92 (4): 1029-34). Soy-containing diet suppresses chronic neuropathic sensory disorders in rats (Anesth Analg. 2001 April; 92 (4): 1029-34); however, rat studies have been inconsistent in showing the neuropathic pain relief from soy protein intake although recent rat studies have shown pain relief heat hyperalgesia has also been demonstrated following consumption a combination of soy fat which was enhanced by intake of soy protein (Perez J, Ware M A, Chevalier S, Gougeon R, Bennett G J, Shir Y. Dietary fat and protein interact in suppressing neuropathic pain-related disorders following a partial sciatic ligation injury in rats (Pain. 2004 October; 111 (3):297-305).

On the other hand, a recent human trial involving soy intake did not shown strong results with respect to pain relief even when people's diets were adjusted to include large amounts of soy (Oct. 3, 2004; CanWest News Service, Charlie Fidelman. Source: CanWest News Service; Montreal Gazette).

It is thus clear that in view of all the available clinical studies, one cannot predict if a derived soymilk product would have a significant blood pressure-lowering effect in hypertensive subjects. It is thus also clear that one cannot predict if a derived soymilk product would have a significant effect on pain relief and treatment or reduction of inflammation.

There is thus a constant need for innovating new compositions which have beneficial health effects to specific health conditions and methods for producing the same. There is also need for new soy kefir product that are more potent that cow milk kefir or soymilk products. There is also a need to provide new anti-hypertensive, anti-inflammation and pain relief compositions.

### SUMMARY

An object of the present invention is to provide a soy fermented product having increased potency.

Another object of the invention is to provide a soy kefir fermented product useful for treating health conditions related to pain, high blood pressure and/or inflammation.

More specifically the objects are achieved by a soy kefir powder obtained by fermenting soymilk with active kefir grains from the Moscow kefir strain. The soy kefir powder of the present invention comprises at least a total isoflavone composition of approximately 0.1-0.4%.

The invention also concerns a method for preparing the soy kefir powder of the present invention. The method comprises the steps of:
a—fermenting soymilk with active Moscow kefir grains under suitable fermentation conditions to obtain a fermentation culture in a ratio ranging between 20/1 to 100/1 (volume/weight) of soymilk to active Moscow kefir grain;
b—separating the active Moscow kefir grains from the fermentation culture obtained in step a) to obtain a fermentation liquid; and
c—spray drying the fermentation liquid obtained in step b) so as to obtain a soy kefir powder.

The invention also concerns a method of pain relief, blood pressure reduction and/or inflammation reduction in a subject in need thereof. The method comprises the step of administering to this subject the soy kefir powder of the present invention.

The present invention has the advantage of providing a soy kefir powder with an improved isoflavone profile compared to regular soymilk, obtained by a method that is significantly less complex and less costly than processes known in the art. Furthermore, the soy kefir powder of the present invention has increased potency over related products derived from other processes. The soy kefir powder of the present invention also has the advantage of being a natural product, it does not cause side effects nor adverse effects. The soy kefir powder of the present invention is thus safe to use by pregnant women or subjects under other medications. The soy kefir powder of the present invention can be taken for prolonged periods of time. Moreover, the soy kefir powder of the present invention is easily accessible to anyone as it may be obtained without the need of a prescription.

### BRIEF DESCRIPTION OF THE FIGURES

FIG. 1. Overlap of spray-dried soymilk capillary zone electrophoresis. Four peaks were observed in the kefir grain fermented soymilk electropherograms that were not visible in the unfermented soymilk while two major peaks observed in unfermented soymilk were absent from fermented soymilk, indicative of the characteristic protein/peptide profile of fermented soymilk.

FIG. 2. shows the study schematic of Example 4.

FIG. 3 is a bar chart showing the differences in improvement of mean scores of SF-36v2 subscales at the endpoint versus baseline. Five-point change in the SF-36v2 health status score is considered as a clinically meaningful change (Frost M H et al. Mayo Clin. Proc. 2002, 77: 488-494; Samsa G. et al., Pharmaco. Economics 1999, 15:141-155; Rowbotham M. C. Pain 2001, 94:131-132).

FIG. 4 is a flow chart illustrating a method for preparing soy kefir powder according to a preferred embodiment of the invention.

### DETAILED DESCRIPTION OF THE INVENTION

### Definitions

By the term “Moscow Kefir grain” it is meant the kefir grain obtained under an exclusive licence from the All-Russia Dairy Institute (ARDI), 35 Lyusinovskaya Street, Moscow, Russia. Table 2 summarizes the composition of the microflora of the Moscow kefir grain.

By the term “soy kefir liquid”, it is meant the liquid obtained by the fermentation of soymilk with the Moscow kefir grains. For instance, such a liquid may be the fermentation culture obtained at step b) of the method according to the present invention.

As used herein, the term “treating” refers to a process by which the symptoms of defined a disorder are alleviated or completely eliminated. Thus, in the context of disorders caused by inflammation, the inflammation symptoms are alleviated or completely eliminated.

By “approximately” it is meant that the value of the composition varies within a certain range depending on the margin of error of the method used to evaluate such composition. For instance, approximately means that the item, parameter or term so qualified encompasses a range of plus or minus 5% of the actual value above and below the value of the stated item, parameter or term. For instance a value of approximately 0.009% may vary between 0.0085 and 0.0095%, a temperature of approximately 19° C. may vary between 18.5 and 19.5° C., a dose of approximately 10 g may vary between 9.5 and 10.5 g.

The term “preventing” refers to a process by which the defined disorder is obstructed or delayed.

By the term “inflammation” is intended, for the purpose of this invention, a localized protective response elicited by injury or destruction of tissues which serves to destroy, dilute or wall off both the injurious agent and the injured tissue, characterized in the acute form by the classical sequence of pain, heat, redness, swelling, and loss of function, and histologically involving a complex series of events, including dilatation of the arterioles, capillaries, and venules with increased permeability and blood flow, exudation of fluids including plasma proteins, and leukocyte migration into the inflammatory focus.

By “reduction of inflammation”, “inflammation reduction” or “anti-inflammation” it is meant the inhibition, that is, arresting the development or further development of clinical symptoms, e.g., mitigating or completely inhibiting active (ongoing) inflammation so as to decrease inflammation, which decrease can include substantially complete elimination of inflammation.

By the term “pain”, it is meant an unpleasant sensation that can range from mild, localized discomfort to agony. Pain has both physical and emotional components. The physical part of pain results from nerve stimulation. Pain may be contained to a discrete area, as in an injury, or it can be more diffuse, as in disorders like fibromyalgia. Pain is mediated by specific nerve fibers that carry the pain impulses to the brain where their conscious appreciation may be modified by many factors. The International Association for the Study of Pain (IASP) defines pain as “an unpleasant sensory and emotional experience associated with actual or potential tissue damage or described in terms of such damage.” Chronic pain has no useful biological function. It can be defined broadly as pain that lasts longer than a month following the healing of a tissue injury; pain that recurs or persists over a period of three months or longer; or pain related to a tissue injury that is expected to continue or get worse. Chronic pain may be either continuous or intermittent. Chronic pain may be back pain, joint pain such as due to arthritis, pain due to surgery, pain due to injury such as sport injuries, accidents injury or any type of injury. Examples of pain are long-standing pain in shoulder (bursitis) and neck pain or pain any other part of the skeletal system. Pain may be due to inflammation such as tendonitis or arthritis. Pain may be also pain in the coccyx area. Pain may be associated with joint replacement surgery.

By the terms “relieving pain” or “relief of pain”, it is meant the relief of pain and discomfort in, but not limited to, joints, bones, muscles and related connectives tissues. The pain may be related to a surgery or a disorder or simply related to a day-to-day type of pain. When measured on a scale from 0 to 5, 0 meaning no pain and 5 meaning symptom at its greatest intensity. By pain relief it is meant a reduction in pain score as assessed by the subject from 5 or 4 to 1 or 0. When assessed with the SF-36v2 scale, a five point change in the scoring meant clinically meaningful pain relief or reduction.

By the term High blood pressure (or hypertension) it is meant in an adult as a blood pressure greater than or equal to 140 mm Hg systolic pressure or greater than or equal to 90 mm Hg diastolic pressure. By “blood pressure reduction”, “blood pressure lowering”, “lowering blood pressure” or “anti-hypertensive” it is meant lowering the blood pressure to a value closer to the normal values recommended by the American heart Association, i.e less than 120 mm Hg systolic and less than 80 mm Hg diastolic. In general a drop of 5 mm Hg in either systolic or diastolic blood pressure is considered clinically significant (Methods of measuring blood pressure at the clinic. 2002. A. P. Follett, F. A. C. Burden, M. L. Burden. Diabetes and Primary Care 4: 19-25).

By the term “subject” it is intended, for the purpose of this invention, any live form that is subject to high blood pressure, inflammation and pain. Examples include, but are not limited to, humans, monkeys, cows, pigs, sheep, goats, dogs, cats, mice, rats, and transgenic species thereof. In a preferred embodiment, the subject is a primate. In an even more preferred embodiment, the primate is a human. Other examples of subjects include experimental animals such as the ones listed above. The experimental animal can be an animal model for a disorder such as hypertension, inflammation, pain.

As used herein, the expression “an acceptable carrier” means a vehicle for containing a soy kefir powder of the present invention. The carrier can be administered to a subject without adverse effects. Suitable carriers known in the art include, but are not limited to, a liquid such as sterile water, drinking water, milk, juice or any drinkable liquid. Carriers may include a solid or creamy food product such as a yogurt, cereals, oatmeal, pudding or any suitable food product in accordance with the present invention.

The term “patients,” as used herein, refers to a subject as defined previously and more preferably a human.

QD is of the Latin language “Quaque die”. When referring to a prescription or to soy kefir powder intake, it means once a day preferably at a regular timing. BIP means twice a day, morning and evening and preferably at regular intervals.

The term “fermenting” as used herein, refers to a bioprocessing process of a chemical change caused by enzymes produced from bacteria, microorganisms or yeasts or amixture thereof, incubated under specific conditions to produce various chemical or pharmaceutical or nutraceutical compounds.

### Soy Kefir Powder of the Invention and Uses Thereof.

According to an embodiment, the present invention relates to soy kefir powder obtained by fermenting soymilk with active kefir grains from the Moscow kefir strain. The soy kefir powder of the present invention comprises at least a total isoflavone composition of approximately 0.1-0.4% (w/w). Advantageously, the isoflavones composition of the soy kefir powder of the present invention is preferably 0.25% (w/w). The isoflavones of the soy kefir powder of the present invention are for instance isoflavones glycosides or aglycones. As contemplated by the present invention, aglycone is preferably selected from the group consisting of: daidzein, genistein and glycetein.

In a preferred embodiment of the invention, the aglycones composition present in the soy kefir powder of the invention is approximately 0.01 to 0.03% (w/w). In the event where the aglycone consists of daidzein, the daidzein composition present therein preferably ranges approximately between 0.006 and 0.020% and more preferably approximately 0.0185% (w/w).

In the case where the aglycone consists of genistein, the genistein composition present therein preferably ranges approximately between 0.003 and 0.01% and more preferably approximately 0.009% (w/w).

The Applicant have surprisingly found that the aglycone concentration of the soy kefir powder of the present invention shows a three-fold and four-fold increase relative to the highest aglycone concentrations observed in unfermented soymilk. Hence, smaller amount of soy kefir powder of the invention only need to be used compared to amounts of unfermented soymilk, which allows for improved bioactivity related to isoflavones to be observed at intakes that are too difficult to reach with soy milk due to the large volumes of milk that would be needed to be consumed.

Another embodiment of the present invention relates to the use of the soy kefir powder of the present invention for lowering pain relief, blood pressure reduction and/or inflammation reduction. Indeed, the inventors have surprisingly found that the soy kefir powder of the present invention is a unique and more potent product than those known in the art.

In a connex embodiment, the present invention provides methods for pain relief, blood pressure reduction and/or inflammation reduction in a subject in need thereof. The methods of the invention comprise the steps of administering to said subject an effective amount of the soy kefir powder of the present invention.

The amount of soy kefir powder of the present invention is preferably a therapeutically effective amount. A therapeutically effective amount of the soy kefir powder of the present invention is the amount necessary to allow the same to perform its role of pain relief, blood pressure reduction and inflammation reduction, without causing overly negative effects in the individual to which the soy kefir powder of the present invention is administered. The exact amount of soy kefir powder of the present invention to be administered will vary according to factors such as the type of disorder being treated, as well as other ingredients which may be given jointly. Suitable dosages will vary, depending upon factors such as the desired effect (short or long term), the route of administration, the age and the weight of the individual to be treated.

The effective amount of soy kefir powder of the invention preferably contemplated in the present invention in order to provide the pain relief effect to an individual in need thereof is preferably an amount ranging from 10 g to 100 g per dose and more preferably approximately 35 g per dose.

The effective amount of soy kefir powder of the invention preferably contemplated in the present invention in order to provide the blood pressure reduction effect to an individual in need thereof is preferably an amount ranging from 10 g to 100 g per dose and more preferably approximately 35 g per dose.

The effective amount of soy kefir powder of the invention preferably contemplated in the present invention in order to provide the inflammation reduction effect to an individual in need thereof is preferably an amount ranging from 10 g to 100 g per dose and more preferably approximately 35 g per dose.

As may be appreciated by a person skilled in the art, the soy kefir powder of the present invention is preferably given to an individual per os. For instance, the soy kefir powder according to the present invention may be administered in a solid or dissolved form to the subject. Liquid vehicles are, but not limited to, water, juice, milk or any other food beverage to the liking or the choice of the subject.

Yet in another preferred embodiment, the soy kefir powder of the present invention may be mixed with solid foods such as cereals, yogurt, pudding or any solid food that may be ingested and is suitable to the subject.

Advantageously, for pain relief, to reduce blood pressure and/or to reduce inflammation, the soy kefir powder of the present invention is preferably administered to subjects in need thereof daily. The soy kefir powder of the present invention may be administrated twice a day and preferably once per day. In yet another preferred embodiment the soy kefir powder of the present invention is administered according to the need of the subject. Hence, the soy kefir powder of the present invention may be administrated every other day or twice a week or according to a suitable regimen. The regimen of administration may thus vary according to the health state of the subject taking the soy kefir powder of the present invention or the therapeutic goal to be achieved. As seen from the examples below, the soy kefir powder of the present invention may have an effect after only 1 day of administration. Hence, the soy kefir powder of the present invention is preferably administered for a period of time ranging from as short as only one day to as long as one year and more preferably for a period ranging from 2 to 4 weeks.

Studies have shown no adverse effects of the intake of the soy kefir powder of the present invention on the health of subjects taking it for prolonged periods of time. Hence, the soy kefir powder of the present invention may also be taken for a period longer than one year.

In order to provide an adequate and regular supply of the potent soy kefir powder of the invention to the subject, the soy kefir powder is preferably used for administration at regular hours. In a preferred embodiment, the spy kefir powder of the invention is given to the subjects in the morning with breakfast. According to another preferred embodiment, the soy kefir powder of the present invention may be given at any time during the day or night. In a further preferred embodiment the soy kefir powder of the present invention is taken at regular recurrent time intervals, such as but not limited to at breakfast every other day or at breakfast every three days, or every 12 hours.

By using the soy kefir powder of the present invention, subjects unexpectedly and advantageously experienced a relief of pain and inflammation reduction. By relief of pain and inflammation reduction, it is meant that the subjects' rating of pain and inflammation on a scale from 0 to 5 (where 5 means symptom at its greatest intensity and 0 means no symptoms, see example 3), is reduced from 5 or 4 to 1 or even zero. Pain relief may also mean a clinically significant reduction on the SF36v2 scoring scale. A clinically significant reduction on the SF36v2 scoring scale is preferably a reduction of 5 points.

By using the soy kefir product according to the invention, subjects have unexpectedly and advantageously experienced blood pressure reduction. By reduction in blood pressure (BP) it is meant a clinically significant reduction. For instance, reduction results in the decrease of at least 5 mm Hg for systolic and diastolic BP or een in the restoration of normal levels of systolic and/or diastolic BP (as recommended by the American Heart Association).

The soy kefir powder of the present invention according to a preferred embodiment, comprises at least one of the following:
a protein composition of approximately of 25-45%,
a carbohydrate composition of approximately of 25-45%,
a fat composition of approximately of 25-45% and
an ash composition of approximately of 9-15%.

In another preferred embodiment, the soy kefir powder of the present invention further comprises the following triglyceride composition:
a saturated fatty acids composition of approximately 2-3.5%; and more preferably approximately 3%,
a monounsaturated fatty acids composition of approximately 3.5-5.5%; and more preferably approximately 4%, and
an N-6 polyunsaturated fatty acids composition of approximately 10-13%, and more preferably approximately 11%.

As one skilled in the art may appreciate, the soy kefir powder of the present invention has the advantage of being a natural health food product or supplement with increased potency relative to unfermented soymilk or fermented cowmilk.

### Method of Preparing the Soy Kefir Powder of the Invention

According to another embodiment, the present invention provides a method for preparing soy kefir powder. Advantageously, the preparation method of the invention offers a soy kefir powder which has at least one biological activity selected from the group consisting of: inflammation reduction, pain relief and blood pressure reduction.

The preparation method comprises a step a) of fermenting soymilk with active Moscow kefir grains under suitable fermentation conditions to obtain a fermentation culture. The soymilk is advantageously in a ratio ranging between 20/1 to 100/1 (volume/weight) of soymilk to active Moscow kefir grain. Indeed, the inventors have surprisingly found that the above mentioned range of soymilk to active Moscow kefir grains provide enough nutrients from the milk for the kefir microbial components for the fermentation to take place and to therefore provide a soy kefir powder with increased potency over related products of the art.

Step b) of the preparation method consists in the separating of the active Moscow kefir grains from the fermentation culture obtained in step a) to obtain a fermentation liquid. Indeed, as kefir grains increase in volume during fermentation, a portion of grains are removed to maintain constant grain-to-milk ratio. Preferably, the grains are removed by coarse sieving or by draining the fermentation liquid. The removed Moscow kefir grains can then advantageously be used as inoculum for fermenting a subsequent batch of soymilk. At this stage, the kefir grains may be preferably lyophilized for long-term storage.

According to a preferred embodiment, a step to refrigerate the fermentation liquid obtained in step b) may be added prior to step c). Advantageously, the fermentation liquid is cooled at a temperature ranging for instance between 2 to 8° C. for about 1 to 5 days. Indeed, the inventors have surprisingly found that this cooling step further potentiate the blood pressure-lowering, pain relief and inflammation relief effect of the soy kefir powder of the present invention.

Step c) of the preparation method consists in the spray drying of the fermentation liquid obtained in step b), or preferably in the spray drying of the refrigerated fermentation liquid as defined above, to form a soy kefir powder of the present invention. This drying step allows removal of any significant amount of alcohol. The fermentation culture is preferably spray-dried at a temperature of 65° C.±13° C. It will be understood that any suitable spray drier known to one skilled in the art may be used in accordance with the preparation method of the present invention.

According to another preferred embodiment, the kefir powder may be then processed to separate from the same agglomerated kefir powder called “chunks”. In such a case, the chunks are thus preferably crushed and then added and mixed to the kefir powder.

As used herein, the term “active Moscow Kefir grains” relates to Moscow kefir grains which are in a ready-to-be-used form suitable for fermentation. For instance, such active Moscow kefir grains are those that had preferably undergone a reactivation step prior to step a) of the preparation method of the invention. In this reactivation step which may be defined as a pre-fermentation step, the Moscow kefir grains are allowed to ferment milk for about 2 to 6 days prior to be introduced to step a) of the preparation method of the invention. Preferred milk used in the reactivation step may be cow milk, soy milk or any other suitable milk known to one skilled in the art.

As may be appreciated by a person skilled in the art, the Moscow kefir grains used in the pre-fermentation step may be in the wet form or in the freeze-dried form. It will be understood that, in the case where the Moscow kefir grains are in the freeze-dried form, the grains are preferably rehydrated according to known methods to one skilled in the art before their introduction to the reactivation step.

It will be understood that a suitable fermentation condition in terms of time contemplated by the present invention is preferably approximately 10 to 24 hours, more preferably approximately 16 to 24 hours and even more preferably approximately 16 hours.

It will be also understood that a suitable fermentation condition in terms of temperature contemplated by the present invention is preferably at room temperature. As used herein, the term “room temperature” refers to a temperature ranging preferably from approximately 19 to 27° C.

It will be further understood that a suitable fermentation condition in terms of pH contemplated by the present invention is preferably between approximately 4.6 and 5.0, and more preferably at approximately 4.8.

Product manufacturing in terms of fermentation and processing has shown excellent reproducibility for bioactivity and product characterization. Stability testing has shown that the soy kefir powder of the present invention is stable preferably when stored at approximately 4° C.

There are no significant variations of vitamin and mineral content following kefir fermentation from the original sourced soymilk; however, an increase in proteolysis leads to an increase in peptides and free amino acids.

### Example 1

### Preparation of Soy Kefir Powder According to a Preferred Method of the Invention

Hundred (100) cases of sterile (UHT) SO NICE Natural soymilk (Soyaworld Inc.), each case consisting of twelve 946 mL Tetrapaks, were obtained (three production lots). Soymilk was stored at 4° C. in a walk-in cold room.

A 150 L Chemap fermentor was used for all production fermentations.

Prior to the first fermentation, the fermentor was cleaned using standard protocols known to one skilled in the art and then steam sterilized at 121° C. using a computer controlled sterilization cycle. The fermentor was equipped for on-line control of temperature and continuous monitoring of pH. The fermentations were run without air addition (anaerobic) and without agitation, except for brief periods during startup and harvesting.

The fermentation substrate consisted of soymilk and dextrose. Except for batches designated K0830A and K0830B, each batch used 9 cases of milk (102 L) plus 2 kg of dextrose. Batches K0830A and K0830B used 90 L of milk plus 1.78 kg of glucose. The temperature controller maintained an optimal fermentation temperature (from 19° C. to 27° C.). The milk and glucose were added to the fermentor manually and then agitated for 2 minutes at 250 rpm prior to addition of the grains (starter culture). The initial starter culture consisted of wet grains plus fermented kefir. In subsequent fermentations the grains consisted of the solids filtered from the previous batch of harvested kefir. After addition of the grains, the agitation was continued at 250 rpm for an additional 2 minutes. At this time the fermentation was considered as started and the pH and temperature were noted.

The fermentation was continued at constant temperature with no agitation for a specified length of time (16 to 24 hours).

At the end of the batch and prior to harvesting, the kefir was agitated at 250 rpm for 2 min and the harvest line was flushed with steam. Since a pump was not used for harvesting, the kefir was removed from the fermentor by gravity flow, aided by 0.5 barr of air pressure introduced into the fermentor headspace.

The harvested kefir was filtered using a custom-made 316 SS cone sieve with 3.2 mm openings. The filtered liquid was collected in a 200 L SS tank and the collected solids retained for addition into the subsequent batch. From the SS tank, the filtered liquid was placed into 19 L plastic pails, sealed airtight with gasketed lids and placed in a walk-in cold room (note: this was a different cold room from that used for storage of the soymilk substrate).

The above-mentioned process can be repeated several times, for instance for a total of 11 fermentation batches.

After completion of all the fermentation batches, the liquid kefir was stored in a walk-in cold room. Spray drying of the kefir was performed using a Niro Atomizer Spray Dryer Model HT-10-530. Each fermentation batch was spray dried separately. The spray drying conditions for each batch were maintained constant by controlling outlet air temperature to between 60-70° C. by adjustment of throughput rate. The time required and solids yield from each spray dried batch were recorded and, after obtaining a sample for analysis, each batch of powder was hermetically sealed in a plastic bag. After all batches had been spray dried, the powder from all batches (except K0817, first fermentation batch) was sieved using a Kason vibrating screen with 2.1 mm hole size. The total mass of large chunks collected by the sieving operation was 11.66 kg or about 23% of the total product yield. The large chunks were crushed using an Urschel™ high speed chopper and then added to the powder. All sieved and crushed powder was blended together for 30 minutes using a double-action ribbon blender. A 500 g sample was obtained for analysis.

The powdered kefir was packaged in hermetically sealed plastic bags. Each bag was weighed and placed in an airtight plastic pail and stored at 4° C. until use.

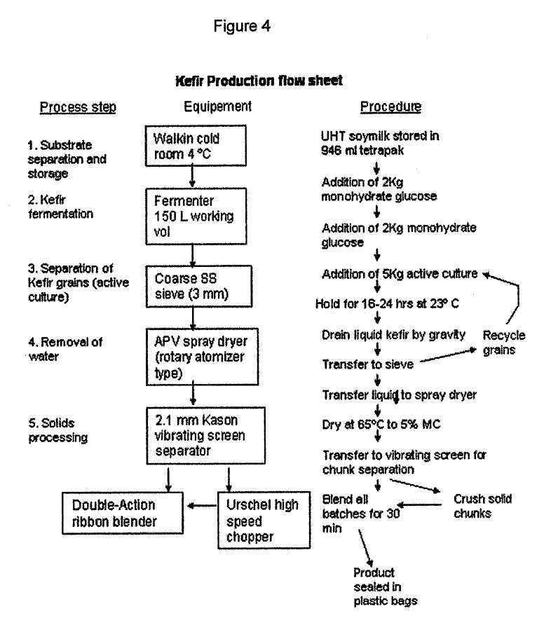

FIG. 4 summarizes the steps followed in this method.

### Example 2

### Use of the Soy Kefir Powder of the Invention for Reducing Pain, Blood Pressure and Inflammation

### Raw Materials Used in Manufacturing

### Source of Soymilk

Soyaworld Inc., Burnaby, BC, Canada.

### Source of Kefir Grains

The All-Russia Dairy Institute (ARDI), 35 Lyusinovskaya Street, Moscow, Russia.

### Fermentation and Processing

Fermentation was done as described in Example 1.

As kefir grains increase in volume during fermentation, a portion of grains are removed to maintain constant grain-to-milk ratio. When fermentation is completed, grains are removed by coarse sieving and used as the inoculum for fermenting a subsequent batch of soymilk.⁵ Alternatively, grains can be lyophilized for long-term storage.¹

Following removal of the grains, liquid kefir is approximately 8% total solids. It is then converted to powder by spray-drying, thus removing any significant amount of alcohol.

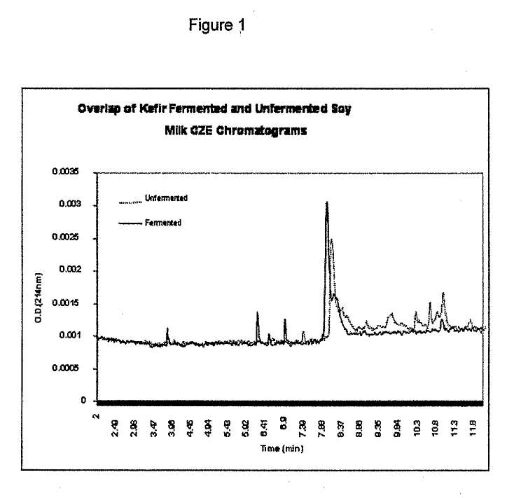

There are no significant variations of vitamin and mineral content following kefir fermentation from the original sourced soymilk; however, a small increase in proteolysis leads to an increase in free amino acids.²³ Indeed, the capillary electrophoretic profile of the Applicant's fermented soymilk demonstrates a protein/peptide profile unique from that of unfermented soymilk (FIG. 1).

### Packaging

Soy kefir powder of the present invention is packaged in 4½″×5½″ paper/foil pouches, each containing 35 grams of powder.

### Case Reports

Human data with soy kefir powder of the present invention is limited to 14 subjects, in which the therapeutic goal of six was Blood Pressure (BP) reduction (Table 4). The results with the soy Kefir Powder of the present invention are indicative of an important therapeutic BP benefit.

One subject with low BP (53/96 mmHg) did not experience any reduction in BP during 2 months of soy kefir powder of the present invention ingestion. In a crossover trial investigating the inflammatory, 500 mL/day of kefir (cow's milk fermented with ARDI kefir grains) was compared to 500 mL/day of placebo (unfermented 2% milk+skim milk powder and water) in 13 human subjects.⁵⁶ Supplements were well tolerated, with both groups demonstrating only mild cramping, constipation and/or bloating within the first week of supplementation. While both treatments contained the same fat content and caloric value, kefir supplementation appeared to induce a weight increase of 0.7 kg (p<0.05), whereas weight remained unchanged with placebo (0.1 kg increase, P>0.05).

Overall, 14 human volunteers have consumed soy kefir powder of the present invention (up to 35 g/day). They experienced no significant adverse events. The human volunteers followed no specific diets. The soy kefir powder of the present invention was used in a liquid vehicle such as water or juice (around 200 ml) or in a dry vehicle such as cereals.

The therapeutic goal of six of the 14 subjects was BP reduction. While clear reductions of BP was demonstrated in patients with hypertension, subjects with normal or low BP did not experience any meaningful reduction in BP while taking the product. There was also an absence of clinically significant adverse effects related to therapy. These encouraging results with the soy kefir powder of the present invention are indicative of an important therapeutic BP benefit not only in terms of blood pressure reduction but also in terms of joint pain relief and anti-inflammatory effect.

Some subjects initially received the Soy Kefir Liquid only whereas others received Soy Kefir Liquid and soy kefir powder of the present invention or soy kefir powder of the present invention only. Subjects received initially the original batch of soy kefir powder of the present invention (Trial 1) and at a later time, most of the same subjects received the batch of soy kefir powder of the present invention to be used in the clinical trial (Trial 2).

The soy kefir liquid used in the present example consists of the liquid obtained by the fermentation of soymilk with the Moscow kefir grains. In other words, such a liquid is the fermentation culture obtained at step b) of the method according to the present invention, whereas the soy kefir powder of the present invention is the powder obtained by spray-drying the above-mentioned soy kefir liquid.

Table 4 shows the results of this study.

### Example 3

### Use of the Soy Kefir Powder of the Invention for Relieving Pain

Case Study Trials with Soy Kefir Powder of the Invention Related to Pain Relief

Overall, 14 human volunteers have consumed soy kefir powder of the present invention (up to 35 g/day). They experienced no significant adverse events. The human volunteers followed no specific diets. The subjects received 35 g of the soy kefir powder of the present invention every day. The soy kefir power was taken by the subjects once a day for periods ranging from 2 to 4 weeks. The soy kefir powder of the present invention was used in a liquid vehicle such as water or juice (around 200 ml) or in a dry vehicle such as cereals. The volunteers also answered a questionnaire daily on the following symptoms: cough, phlegm, joint pain, digestive disturbance, fatigue, stress, depression, bowel irregularity, sleep disturbances and agitation. The answers of the volunteers were collected starting three days before the starting point of the trial, and then daily during the trial. The volunteers rated each symptom on a scale from 0 to 5, with 0 being symptom free, and 5 meaning symptom at its greatest intensity. Table 5 shows a compilation of the results of this study. The daily ingestion of soy kefir powder of the present invention had a clear effect on relieving pain.

### Example 4

### Safety of Soy Kefir Powder of the Invention

### Adverse Reactions

In a crossover trial demonstrating the inflammatory property of cow's milk kefir, 500 ml/day of kefir (cow's milk fermented with ARDI kefir grains) was compared to 500 ml/day of placebo (unfermented 2% milk+skim milk powder and water) in 13 human subjects. Supplements were well tolerated, with both groups demonstrating only mild cramping, constipation and/or bloating within the first week of supplementation.

A double blinded randomized controlled cross-over clinical trial was performed by the applicant that assessed whether intake of the Soy Kefir has health benefits in human subjects in terms of hypertension and anti-inflammatory effects was conducted by the clinical research organization, Ethica Inc. No adverse reactions as determined by examination of routine serum chemistry were shown, i.e., SMAC-24—total bilirubin, creatinine, glucose, uric acid, sodium, potassium, BUN, chloride, CO₂, calcium, phosphorus, magnesium, total protein, albumin, alkaline phosphatase, AST, ALT, GGT, CK, LDL, cholesterol, HDL, triglycerides, iron, and specialized serum assays: aldosterone, renin, IL-6, and CRP. No serious adverse events were observed although some mild adverse events in terms of gastrointestinal events were noted; however, they were in a range of incidences of adverse effects that are very common in such studies. Probiotics such as the soy kefir powder of the present invention commonly show initially higher incidences of gastrointestinal upset. The incidence of gastrointestinal events was markedly lower in the second phase of the cross-over designed study, which was attributed to the ability of the patients to tolerate a higher intake of proteins in the second phase of the study.

US patent publications 20020182274 and 20030008023 described that highly concentrated, filtered extracts derived from soymilk fermented with bacteria and/or yeasts showed no adverse effects when provided to human subjects (i.e., infants, asthmatic children, pregnant and lactating mothers, women undergoing surgery). These same extracts had also undergone acute and chronic toxicity studies in rodents showing no signs of toxicity. The extracts were found to be non-mutagenic in Ames test, they do not cause in vitro mammalian cell chromosomal damage, nor did they induce micronuclei in bone marrow cells in ICR mice.

### Contraindications

MAO inhibitors: Fermented soy products contain significant amounts of tyramine. Tyramine, an indirect sympathomimetic, is known to cause hypertensive reactions in patients receiving MAOI therapy. Therefore, individuals receiving MAOI therapy should avoid co-administration with fermented soy products, including the soy kefir powder of the present invention.

### Precautions

Soy protein allergy: Individuals with soy protein allergies should avoid consumption of soy products, including the soy kefir powder of the present invention.

Phenylalanine metabolic disorders: Therefore, individuals with phenylalanine metabolic disorders such as phenylketouria should avoid the consumption of soy protein products, including the soy kefir powder of the present invention.

Hypothyroidism: High levels of soy isoflavones may inhibit thyroid hormone synthesis by competing for the plasma iodine used in their production. Thus, individuals should avoid taking of soy protein products, including the soy kefir powder of the present invention, within a few hours of taking thyroid medication.

### Objectives

To evaluate 24-hour blood pressure control and frequency of adverse effects in patients with mild to moderate hypertension treated with soy kefir powder of the present invention.

### Design

Single-center, double-blind, randomized, crossover. Following a 2-4 week Placebo Run-In, eligible subjects were randomized to either soy kefir powder of the present invention (35 gram/QD) or placebo and will enter Treatment Period I. After 4 weeks of treatment, subjects enter a 4-week Washout Phase, then crossover into Treatment Period II where they received study medication alternate to that received in the previous Treatment Phase. Serum sampling and 24-Hour ABPM was performed at Baseline and at the completion of each Treatment Phase.

### Population

Subjects with Stage I or Stage II essential hypertension (SBP=140-180 mmHg) with a Mean Daytime SBP (ABPM) of >135 mmHg

### Sample Size

20 subjects

### Treatments

Soy kefir powder of the present invention (35 gram/QD) or matching placebo

### Efficacy Data

24-Hour ABPM measurements, Office BP measurements, serum levels of aldosterone, renin, CRP and IL-6

### Safety Data

Physical examinations, clinical laboratory parameters (SMAC-24), 12-lead ECG, and adverse event reporting

### Statistical Procedures

Adjusted mean end-treatment change from Baseline in blood pressure variables (ABPM and Officemeasurements) were compared between the soy kefir powder of the present invention and placebo groups by ANCOVA. Baseline measurements were used as covariate. The χ² test was used to compare categorical demographic and adverse events data. Multiple regression was used to examine the association of changes in blood pressure and laboratory parameters (i.e., aldosterone, renin). Standard end-treatment change from Baseline analyses were employed for CRP and IL-6.

### Study Duration

Study duration is approximately 14-16 weeks.

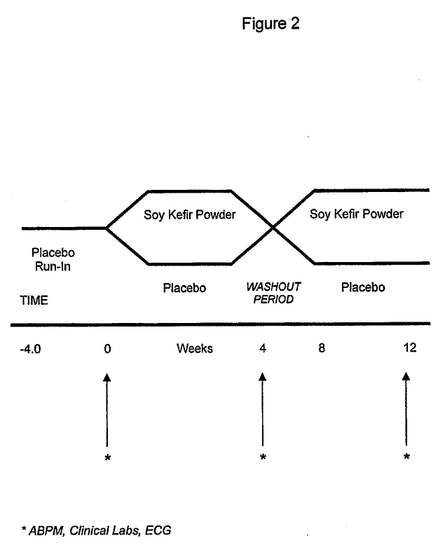

The study schematics are shown in FIG. 2.

### 1 General Information

### 1.1 Introduction

The Soy kefir powder of the invention is consumed after reconstitution in juice or water. While controlled clinical trials have yet to confirm the clinical utility of most uses, studies have demonstrated that kefir beverages and/or fermented milk products possess potent BP lowering effects with few if any adverse effects.⁹²⁻⁹⁵ The therapeutically active anti-hypertensive agents in soy kefir powder of the present invention appear to be bioactive peptides with ACE inhibition properties. Various ACE-inhibitory peptides have been described originating from different food sources released after hydrolytic and/or fermentation processes.^{24,25} While it is well known that peptides derived from fermented milk proteins demonstrate various degrees of ACE inhibition,²⁶⁻²⁹ the use of genuine Russian Kefir grains in the applicant's fermentation process results in a unique and typically more potent product than those derived from other processes.

Compared to milks cultured solely with a one to three bacterial strains, Russian Kefir grains contain numerous strains of bacteria and yeast (table 2) fermentation with which produces a significantly wider variety of ACE-inhibiting components.^{28,2,3} Furthermore, opposed to fermented cow's milk, fermented soymilk is rich in isoflavones.

Isoflavones have been demonstrated to possess anti-hypertensive, anti-inflammatory, and anti-oxidative properties. Therefore, the soy kefir powder of the present invention offers multiple therapeutic modalities benefiting antihypertensive therapy.

Human data with the soy kefir powder of the present invention is limited to 14 subjects, in which the therapeutic goal of six was BP reduction. While clear reductions of BP was demonstrated in patients with hypertension, subjects with normal or low BP did not experience any meaningful reduction in BP while taking the product. There was also an absence of clinically significant adverse effects related to therapy. Albeit preliminary, these encouraging results with the soy kefir powder of the present invention are suggestive of an important therapeutic BP benefit.

The current study investigated the antihypertensive effects of the Soy Kefir Power using 24-Hour ABPM. To provide biochemical confirmation of classical angiotensin-renin system inhibition, serum levels of aldosterone and renin were monitored. Additionally, as ACE inhibition could modify various inflammatory actions in the vascular wall,^{37,40} treatment effects on IL-6 and C-RP was also monitored.

### 1.2 Good Clinical Practice

All activities performed within the scope of this study comply with recognized Good Clinical Practice guidelines and applicable regulatory requirements.

### 1.3 Study Population

20 subjects with mild to moderate essential hypertension participated in this study.

### 2 Objectives and Purpose of Study

To evaluate 24-hour blood pressure control and frequency of adverse effects in patients with mild to moderate hypertension treated with soy kefir powder of the present invention according to the invention.

### 3 Study Design

The study was carried as a single-center, double-blind, randomized, crossover study. After a 2-4 weeks Placebo Run-In Phase, eligible subjects were randomized to either soy kefir powder of the present invention (35 gram/QD) or placebo and entered Treatment Period I. Following 4 weeks of treatment, subjects' blood pressure was allowed to stabilize during a 4-week Washout Phase. Subjects then crossed over into Treatment Period II where they received study medication alternate to that received in the previous Treatment Phase. Serum sampling and 24-Hour ABPM were performed at Baseline and at the completion of each Treatment Phase.

### 4 Selection and Withdrawal of Subjects

### 4.1 Inclusion Criteria

(i) Outpatient, male or female subjects, of any race, between 18 and 80 years of age. Female subjects of childbearing potential had a negative urine pregnancy test result at Baseline and practiced a reliable method of contraception.

A female is considered of childbearing potential unless she is:
postmenopausal for at least 12 months prior to study drug administration;
without a uterus and/or both ovaries; or
has been surgically sterilized for at least 6 months prior to study drug administration.

Reliable methods of contraception are:
hormonal methods or intrauterine device in use at least 30 days prior to study drug administration;
barrier methods plus spermicide in use at least 14 days prior to study drug administration; or
sexual abstinence as a lifestyle.
   (ii) Subjects with Stage I or Stage II essential hypertension at Baseline (SBP=140-180 mmHg).
   (iii) Mean Daytime SBP (ABPM) of >135 mmHg.
   (iv) Subjects who are able to understand the requirements of the study, including signing Informed Consent.

### 4.2 Exclusion Criteria

(i) Female subjects who are pregnant (positive urine pregnancy test), are planning to become pregnant during the study period, have an infant they are breast-feeding, or who are of childbearing potential and not practicing a reliable method of birth control.
(ii) Subjects with secondary, malignant, or accelerated hypertension.
(iii) Subjects with Stage III hypertension (SBP >180 mmHg).
(iv) Subjects with any history of cerebrovascular accident or transient ischemic attack.
(v) Subjects with a history of angioedema.
(vi) Subjects with hyperkalemia (serum potassium >5.5 mmol/L.
(vii) Subjects with phenylalanine metabolic disorders (i.e., phenylketouria).
(viii) Subjects who have experienced a myocardial infarction and/or unstable or stable angina within the last 12 months.
(ix) Subjects with Diabetes Mellitus (Type I or Type II).
(x) Subjects with a history of hypertensive encephalopathy.
(xi) Subjects with a history of significant valvular heart disease.
(xii) Subjects with a history of clinically significant arrhythmia.
(xiii) Subjects with a known history of congestive heart failure and/or an ejection fraction <50%.
(xiv) Subjects with a history of clinically significant liver impairment (i.e., ALT or AST >3× upper level of normal).
(xv) Subjects with a calculated serum creatinine clearance <70 mL/min as determined by the Cockcroft Gault formula: ((140−age in yrs)×weight in kg)/(814.464×mmol creatinine). If female, the product of this equation was multiplied by 0.85.
(xvi) Subjects with sensitivity or allergy to soy protein, lactose, milk protein, or other milk component(s).
(xvii) Subjects with terminal illness or chronic infection.
(xviii) Subjects currently taking prohibited medication(s) indicated in Section 5.4.
(xix) Subjects with evidence of recent alcohol/drug abuse, or acute medication overuse.
(xx) Subjects planning or requiring any surgeries during the study.
(xxi) Subjects with a history of poor cooperation, non-compliance with medical treatment, or unreliability.
(xxii) Subjects participating in an investigational drug study or who have participated in an investigational drug study within 30 days of the Baseline. Any waiver of the above Inclusion/Exclusion criteria must be agreed to by the Sponsor and Investigator, and be approved by the Institutional Review Board.

### 4.3 Withdrawal of Subjects

It was the right and duty of the Investigator to discontinue the study participation of a subject when the subject's health or well-being was threatened by continuation in the study. Such subjects were withdrawn from the study and not continued under a modified regimen. The following are circumstances that would result in the subject's discontinuation from the study:
the subject experiences a serious adverse event rendering them unable to continue study participation;
the subject is unable to physically or mentally tolerate the use of the study medication;
an exclusion criteria becomes apparent at any time during the study; or
the subject voluntarily withdraws.

A subject who is withdrawn from the study prior to initiation of treatment may be replaced.

### 5 Treatment of Subjects and Follow-Up

### 5.1 Study Procedure

The study procedure is described in Table 25.

### 5.1.1 Description of Study Days

### Screening Phase (Visit 1A-1D, Weeks −4 to 0)

At Visit 1A the subject completed the following:
Informed consent
Inclusion/exclusion criteria
Medical history
Physical examination
Urine pregnancy test (if applicable)
Office BP Measurement (Supplement I)
Concomitant medications
Previous medications
Record medications taken within the last 1 month
Prohibited Medication Washout
Study Treatment
Dispense 4-week supply of single-blind placebo
Appointment for Visit 1B in approximately 1 week.

At Visit 1B the subject's Office BP was recorded and subject qualification were considered. The subject qualified to immediately complete all procedures outlined for the Visit 2 (Baseline) if, a) SBP is 140-180 mmHg, and b) it is the Investigator's opinion that the subject's mean daytime SBP (ABPM) would be >135 mmHg. If the subject did not qualify, he/she attended Visit 1C in approximately 1 week, and Visit 1D approximately 1 week after that, if necessary. Subjects failing to qualify by Visit 1D was considered as screen failures and will be discontinued from further study participation.

### Visit 2 (Baseline, Week 0)

The following procedures was performed at a Qualifying Screening Visit (i.e., Visit 1B, 1C, or 1D):
Urine pregnancy test (if applicable)
Office BP measurement
Inclusion/exclusion criteria
12-lead ECG
Clinical laboratory tests (Supplement III)
Concomitant medications
Adverse events
24-Hour ABPM (Supplement II)
Randomization to treatment
Study medication
Dispense 4-week supply of study medication according to subject's TAN
Appointment for Visit 3 (Week 2)

### Visit 3 (Week 2, Safety Follow-Up)

Office BP measurement
Concomitant medications
Adverse events
Appointment for Visit 4 (Week 4)

### Visit 4 (Week 4)

Office BP measurement
Physical examination
12-lead ECG
Clinical laboratory tests
Concomitant medications
Adverse events
24-Hour ABPM
Appointment for Visit 5 (Week 6)

### Visit 5 (Week 6, Safety Follow-Up)

Office BP measurement
Concomitant medications
Adverse events
Appointment for Visit 6 (Week 8)

### Visit 6 (Week 8)

Urine pregnancy test (if applicable)
Office BP measurement
Concomitant medications
Adverse events
Study medication
Dispense 4-week supply of study medication according to subject's TAN
Appointment for Visit 7 (Week 10)

### Visit 7 (Week 10, Safety Follow-Up)

Office BP measurement
Concomitant medications
Adverse events
Appointment for Visit 8 (Week 12)

### Visit 8 (Week 12)

Office BP Measurement
Urine pregnancy test (if applicable)
Physical examination
12-lead ECG
Clinical laboratory tests
Concomitant medications
Adverse events
24-Hour ABPM

### 5.1.2 Dietary Restrictions

Subjects were instructed to present to study visits having abstained from eating, smoking, and/or consuming caffeinated beverages for at least 30 minutes prior. Clinical laboratory tests require an 8-hour fast; therefore, subjects were instructed to fast overnight for Visits 2, 4, and 8.

Subjects should refrain from eating for 1-hour after the commencement of ABPM procedures.

Subjects were instructed to make all possible efforts to maintain a consistent diet throughout the study.

### 5.2 Study Treatment

### 5.2.1 Details of Study Treatment

Active: The soy kefir powder of the present invention was provided in 35 gram sachets. The Soy Kefir powder of the present invention appears off-white to light tan in color and is composed of approximately 43% protein, 26% carbohydrate, 18% fat, 8% moisture, and 5% ash.

Placebo: Placebo matching the colour, taste, texture and smell of soy kefir powder of the present invention were formulated using cow's milk casein, dark malt extract, dextrose, coffee whitener and cream of tartar. It was packaged in 35 gram sachets and was identical in appearance to soy kefir powder of the present invention.

### 5.2.2 Preparation of Study Treatment

To prepare the study medication for consumption, a single sachet was mixed in at least 250 ml of fruit juice. The type of fruit juice was left to the discretion of the subject and, if the subject prefers, he/she may use a greater volume of juice. However, the subject was instructed to ensure that the entire dose is consumed immediately upon mixing.

### 5.2.2 Dosage Schedule

Study medication is to be consumed in the morning, between 7:00 AM and 9:00 AM. On study visit days where 24-Hour ABPM is performed, subjects were instructed to present at the clinic prior to consuming study medication. They were also instructed to not take any subsequent dose of study medication until the completion of the ABPM.

### 5.2.3 Treatment Assignment

The study medication was administered only to subjects included in this study following the procedures set out in the Study Protocol.

All subjects who have signed an ICF received a 2-digit subject number, starting at 01. This subject number was used to identify the subject throughout the study. As subjects qualify for the study, they were randomized to a specific treatment sequence, utilizing treatment assignment numbers (TANs). Therefore, TANs are not the same a subject numbers.

A randomization schedule was generated by ethica Clinical Research Inc. This schedule linked sequential numbers (TANs) to treatment codes allocated at random. The schedule was prepared on a balanced 1:1 basis. Eligible subjects were randomized to the study treatment sequence in accordance with the randomization schedule. The next eligible subject received the lowest available TAN. The Investigator documented the TAN on the case report form (CRF).

Subjects withdrawn from the study retain their subject number and their TAN, if already allocated. New subjects must always be allotted a new subject number and, if applicable, a new TAN.

### 5.2.4 Blinding, Packaging, Labeling and Storage

Study medication kits were individually prepared for each TAN by ethica Clinical Research Inc.

The identity of the study medications was blinded and packaged according to the randomization schedule and supplied to the Investigator in boxes. An individual box was provided for each TAN, with each TAN box containing 3 smaller boxes, one for each study visit requiring medication dispensation. Each smaller box contained an appropriate number of blinded sachets of study medication for treatment duration.

All boxes and sachets of study medication were individually labeled. Labeled information included study number, TAN, mixing instructions, and any required regulatory statements.

The investigator was provided with a set of sealed envelopes; one envelope for each TAN. The identity of the medication (treatment sequence) for the TAN was stated on a card inside the envelope. If it was medically imperative to know what study medication the subject was receiving, the Investigator or authorized person opened the envelope and exposed the blinded information. The Investigator or the person who broke the blind recorded the date and the reasons for doing so in the CRF and in the subject's medical records. In such cases, treatment with the study medication was stopped and ethica Clinical Research Inc. was contacted to determine whether the subject should be withdrawn from the study. Whenever possible, ethica Clinical Research Inc. was contacted before the blind was broken.

Study medication was kept refrigerated.

### 5.2.5 Supplies and Accountability

The Investigator or pharmacist inventoried and acknowledged receipt of all shipments of study medication. All study medication was kept in a locked area with access restricted to designated study personnel. The study medication was stored in accordance with the manufacturer's instructions. The Investigator or pharmacist also kept accurate records of the quantities of study medication dispensed, used, and returned by each subject. The site monitor periodically checked the supplies of study medication held by the investigator or pharmacist to ensure accountability of all study medication used. At the conclusion of the study, all unused study medication and all empty sachets were returned to ethica Clinical Research Inc.

### 5.2.6 Compliance

Subjects were instructed to bring their study medication to every visit. Compliance was assessed by sachet counts. Details were recorded on the CRF.

### 5.3 Concomitant Treatment/Medication

All efforts were made to keep the current medication use constant throughout the study; however, modifications were tolerated as long as they are captured in the CRF. Other necessary therapies that did not interfere with the response to treatment were provided to the subject at the discretion of the Investigator. The use of any concurrent medication, prescription or over-the-counter drug was recorded in the CRF along with the reason the medication was taken.

### 5.4 Prohibited Medications

Concurrent forms of treatment that affect blood pressure or interfere with blood pressure therapies were not be allowed. The Investigator used his best clinical judgment in determining whether it is safe to discontinue current therapies, and if a particular medication should be gradually tapered rather than abruptly discontinued. Subjects were prohibited from taking the following medication(s) while they participate in this study:
Any antihypertensive therapy (i.e., ACE inhibitors, beta-blockers, diuretics, CCBs, etc.);
Non-steroidal anti-inflammatory drugs (NSAIDS);
MAO Inhibitors;
Herbal therapies that can affect BP (i.e., St. John's Wort, licorice supplements, ephedra, etc.);
Cox-2 inhibitors.

### 6 Assessments of Efficacy

### Primary Efficacy Endpoint:

Mean daytime SBP as measured by ABPM between 6:00 AM and 10:00 PM (15 min. intervals).

### Secondary Efficacy Endpoints:

ABPM measurements (daytime readings were taken at 15 min. intervals, nighttime readings at 30 min. intervals):
Mean daytime DBP (6:00 AM to 10:00 PM);
Mean daytime HR (6:00 AM to 10:00 PM);
Mean daytime mean arterial pressure (6:00 AM to 10:00 PM);
Mean nighttime DBP (10:00 PM to 6:00 AM);
Mean nighttime SBP (10:00 PM to 6:00 AM);
Mean nighttime HR (10:00 PM to 6:00 AM);
Mean nighttime mean arterial pressure (10:00 PM to 6:00 AM);
Mean 24-hour DBP;
Mean 24-hour SBP:
Mean 24-hour HR;
Mean 24-hour Mean Arterial Pressure.
Office Measurements:
DBP, SBP, HR.
Serum levels of IL-6, C-RP, renin, and aldosterone.

### 7 Laboratory Parameters

Routine Serum Chemistry: SMAC-24—total bilirubin, creatinine, glucose, uric acid, sodium, potassium, BUN, chloride, CO₂, calcium, phosphorus, magnesium, total protein, albumin, alkaline phosphatase, AST, ALT, GGT, CK, LDL, cholesterol, HDL, triglycerides, iron Specialized Serum Assays: aldosterone, renin, IL-6, CRP All serum assays were performed by a contract clinical laboratory (Supplement III) according to standard known laboratory methods.

### 8 Assessments of Safety

### 8.1 Safety Assessments

Adverse events were based on Investigator and subject assessments of signs and symptoms, ECG, physical examinations, and clinical laboratories.

Throughout the study, subjects were monitored for signs and symptoms of adverse events. An adverse event is any pathological or unintended change in the structure, function or chemistry of the body that occurs during the study, irrespective of causality, including any illness, injury, toxicity, sensitivity or sudden death. The condition must either not be present pre-study or must worsen in either intensity or frequency during the study.

A serious adverse event is any untoward medical occurrence, that, at any dose:
results in death;
is life-threatening
requires in-patient hospitalization or prolongation of existing hospitalization
results in persistent or significant disability/incapacity; or
is a congenital anomaly/birth defect.

An unexpected adverse event is any adverse event, irrespective of causality, that is not identified in nature severity or frequency in current literature on the test product.

### 8.2 Reporting Requirements

8.2.1 Serious and/or Unexpected Adverse Events

Any serious or unexpected adverse event occurring in this study were reported to ethica Clinical Research Inc. within 24 hours of awareness of the event.

### 8.2.2 Adverse Event Reporting

All adverse events, including serious and unexpected adverse events, were recorded by the Investigaton. Investigator was required to describe the adverse event, onset date, duration, severity, the course of action taken, if any, as well as any pertinent data necessary to allow a complete evaluation of the adverse event. For serious and/or unexpected adverse events, an additional form was completed.

### 8.2.3 Follow-Up and Final Reports

Subjects who have had a serious adverse event were followed clinically until all parameters, including laboratory values, have either returned to normal or are otherwise explained. If death was the outcome of the event on the initial Adverse Event Report, a Follow-up/Final Report, including autopsy report, when performed, was completed.

### 9 Statistics

### 9.1 Sample Size

The sample size estimation is based on the following assumptions: δ=10 mmHg in mean daytime SBP, Standard deviation: 9.0 mmHg, α=0.05 and β=0.10

In order to be able to detect a clinically significant difference of 10 mmHg between treatment groups in the ABPM measurement of mean daytime SBP, and assuming a 10% dropout rate, it was necessary to enroll 20 subjects into this crossover study.

### 9.2 Analyses

Efficacy and safety analyses were conducted on an intent-to-treat basis. All subjects who received study medication were included in the analysis.

Adjusted mean end-treatment change from Baseline in blood pressure variables (ABPM and Office measurements) were compared between the soy kefir powder of the present invention and placebo groups by ANCOVA. Baseline measurement was used as covariate. The χ² test was used to compare categorical demographic and adverse events data. Multiple regression was used to examine the association of changes in blood pressure and laboratory parameters (i.e., aldosterone, renin). Standard end-treatment change from Baseline analyses was employed for CRP and IL-6.

### Supplement I

### Office Blood Pressure Procedure

Subjects were instructed to refrain from smoking or taking caffeine or food during the 30 minutes before measurement. Subjects were seated quietly for at least 5 minutes in a chair, with feet on the floor, and arm supported at heart level.

Auscultatory readings of blood pressure were performed using a properly calibrated mercury sphygomanometer and an appropriate-size cuff (cuff bladder encircling at least 80 percent of the arm). Systolic blood pressure is the point at which the first of two or more sounds is heard (phase 1), and diastolic blood pressure is the point before the disappearance of sounds (phase 5).

A total of three sequential readings were taken. The cuff was allowed to completely deflate for at least 1 minute between readings.

### Supplement II

### 24-Hour Ambulatory Blood Pressure Monitoring Procedures

24-hour ambulatory blood pressure monitoring was performed using the Spacelabs 90207 (Spacelabs, Inc., Redmond, Wash.). The device was programmed to inflate and record blood pressure every 15 minutes during daytime (6:00 AM to 10:00 PM) and every 30 minutes during nighttime (10:00 PM to 6:00 AM). The cuff ladder was of an appropriate size, encircling at least 80 percent of the arm.

Subjects wore the device for a continuous 24-hour period and recorded daily activities (i.e., meals, sleep, exercise, concomitant medication dosings, etc.) in an activity log.

### Monitoring Days:

To minimize intra-subject variability, all ABPM procedures for a particular subject were performed on similar type days (i.e., weekend, workday).

Training: Subjects were appropriately educated on the procedure and were provided with detailed written instructions.

Study Treatment Subjects presented to the clinic in the morning prior to taking their dose of study medication. The study medication was consumed on an empty stomach immediately after the 24-hour ambulatory monitor is placed and the first blood pressure reading is obtained (approximately 7:00-10:00 AM). Subjects abstained from eating for at least one hour from the first reading. The subsequent dose of study medication was not taken until a complete 24-hour period had elapsed.

### Supplement III

### Contract Laboratory Procedures

A contract laboratory was provided analytical services for all laboratory samples obtained in this clinical study. Individual study visit kits were provided to the site.

A clinical study manual, which contained detailed instruction for the collection, processing and shipment of samples, as well as telephone support were provided to the site by the laboratory. All kits, requisitions, and sample tube labels were bar coded.

Fasting blood samples (8-hour fast) were collected

The following laboratory parameters were measured:
SMAC-24 Panel: total bilirubin, creatine, glucose, uric acid, sodium, potassium, BUN, chloride, CO₂, calcium, phosphorus, magnesium, total protein, albumin, alkaline phosphatase, AST, ALT, GGT, CK, LDL, cholesterol, HDL, triglyceride, iron, Aldosterone, Renin, IL-6, CRP.

### LIST OF ABBREVIATIONS USED IN THIS STUDY

ABPM—ambulatory blood pressure monitoring
ACE—angiotensin converting enzyme
ACEI—angiotensin converting enzyme inhibitor
AI—angiotensin I
AII—angiotensin II

### ARDI—All-Russia Dairy Institute

BP—blood pressure
CCB—calcium channel blocker
CRF—case report form
CRO—contract research organization
CRP—C-reactive protein
CZE—Capillary zone electrophoresis
DBP—diastolic blood pressure
ECG—electrocardiogram
g/—gram
g/day—grams per day
GABA—gamma amino butyric acid

### GCP—Good Clinical Practice

### GRAS—Generally Regarded As Safe

### HPLC—High Pressure Liquid Chromatography

### IB—Investigator Brochure

### IC50—Inhibitory Concentration 50%

### ICF—Informed Consent Form

### ICH—International Conference on Harmonisation

IL-1-interleukin-1
IL-6-interleukin-6
IRB—institutional review board
kg—kilogram
MAO—monoamine oxidase
MAOI—monoamine oxidase inhibitor
mg—milligram
mL/day—millilitres per day

### NHP—Natural Health Product

SBP—systolic blood pressure

### TAN—Treatment Assignment Number

TNF—tumour necrosis factor

### Results

As shown in Table 6, no serious adverse events were recorded and the number of adverse events outcomes observed in the clinical trial was in the normal range. There was no statistical increase in adverse events with soy kefir powder of the present invention intake.

As shown in Table 7, normal blood levels of the grammaglutamyltransferase (GGT) were observed indicating no liver damage occurred with soy kefir powder of the present invention intake.

As shown in Table 8, normal blood levels of the liver enzyme aspartate aminotransferase (AST) were observed indicating that no liver or heart damage occurred with soy kefir powder of the present invention intake.

As shown in Table 9, normal blood levels of bilirubin were observed indicating no liver damage occurred with soy kefir powder of the present invention intake.

As shown in Table 10, normal blood levels of creatine kinase (CK) were maintained indicating no muscle breakdown was associated with soy kefir powder of the present invention intake.

As shown in Tables 11 and 12, normal blood levels of creatinine were observed and creatinine clearance was normal indicating no kidney damage occurred with soy kefir powder of the present invention intake.

As shown in Table 13, normal blood levels of the gammaglutamyltransferase (GGT) were observed indicating no liver damage occurred with soy kefir powder of the present invention intake.

As shown in Table 14, blood levels of blood urea nitrogen were observed to be normal indicating no kidney damage occurred with soy kefir powder of the present invention intake.

### Example 5

### Soy Kefir Powder of the Invention And Chronic Pain Relief

### Introduction

A survey found that 89% of Americans age 18 and older have pain at least once a month and 15% of them have severe pain monthly (Gallup, 1999). Among people aged 65 and older, 55% have pain daily suggesting that chronic pain likely affects many millions of people, with profound consequences on activities of daily living.

The first step in pain relief is usually common oral pain relievers such as aspirin and acetaminophen, and non-steroidal anti-inflammatory drugs (NSAIDs). Chronic users of non-steroidal anti-inflammatory drugs (NSAIDs) have an increased risk of bleeding and damage to their small intestines. Each year in the USA, the side effects of long-term NSAID use cause nearly 103,000 hospitalizations and 16,500 deaths. The COX-2 inhibitors includes ViOXX™, Celebrex™ and Bextra™, have been popular medications used to stop pain and inflammation, particularly in association with arthritis and menstrual pain. Merck withdrew ViOXX™ because of an increased risk of serious cardiovascular events, including heart attacks and strokes among study patients taking ViOXX™. Bextra™, which relieves symptoms of arthritis, and menstrual discomfort was withdrawn in Canada and USA due to similar safety concerns. Celebrex™ is used to relieve the symptoms of osteoarthritis and rheumatoid arthritis in adults; however, recently the National Cancer Institute (NCI) has stopped drug administration in an ongoing clinical trial investigating a new use Celebrex™ to prevent colon polyps because of an increased risk of cardiovascular events in patients taking Celebrex™ versus those taking a placebo.

If NSAID medications do not control pain, opioids are used. Opioid medications are commonly used in the treatment of chronic pain, but they can further complicate the management of chronic pain, possibly worsening pain through increased tolerance and decreasing pain facilitation. For most common chronic pain, opioids could do more harm than good (Schofferman, 1993) as the use of opioids for chronic pain is associated with poor treatment outcomes (Halpern and Robinson, 1985). Neuropathic pain is particularly unresponsive to opioids. Because of the generally poor response of neuropathic pain to opioids, some authors state that the condition usually should not be treated with these agents (Sindrup, 2002).

A variety of studies indicate that a soy diet may reduce neuropathic pain in an animal model of partial nerve injury produced by tightly ligating ⅓-½ of the sciatic nerve 57 (PSL model) (Shir et al., 2001). The beneficial effects of soy protein on PSL may be related to the reduction in inflammation. Inflammation may contribute to chronic pain states such as neuropathic pain, as proinflammatory cytokines and oxidants produced at the site of nerve injury may be involved with sensitization of nociceptors and hyperalgesia (Wagner et al., 1998). Neuropathic pain behaviors are reduced with anti-cytokine treatment (Wagner et al., 1998). Dietary consumption of soy protein isolate significantly reduces the carrageenan-induced production of TNF-alpha in macrophages (Yagasaki et al., 2001) and decreases the degree of edema and thermal hyperalgesia following injection of complete Freund's adjuvant (Tall and Raja, 2002). Recent rat studies have also shown pain relief from thermal hyperalgesia following consumption a combination of soy lipids that was enhanced by intake of soy protein (Perez et al., 2004). Soy lipids have also been implicated in pain relief as rats fed soybean oil had an elevated pain threshold (Yehuda et al., 1986). Other bioactive components in soy could include isoflavones such as genistein that possess anti-inflammatory properties (Sadowska-Krowicka et al., 1998). Genistein has also been shown to inhibit lipopolysaccharide-induced production of the proinflammatory cytokines TNF-alpha, IL-1 alpha, and IL-6 in mixed glia, microglia- or astrocyte-enriched cultures (Kong et al., 1997). Another potential bioactive component is the soluble unique kefir polysaccharide, kefiran, as a recent study has indicated that oral intake of kefir grains induce anti-inflammatory effects in rats (Diniz et al., 2003).

### Case Studies

Investigations by the applicant of clinical parameters affected by the intake of the soy kefir powder of the present invention (35 grams/day) or non-filtered liquid soy kefir (200-400 ml/day) were carried out with case studies of patients with chronic joint pain and neuralgia. More potent pain relieving effects were observed with the soy kefir powder of the present invention as compared to the non-filtered liquid soy kefir. A total of 8 subjects were tested for chronic pain relief and all showed significant improvements in relief from chronic pain that included bursitis, rheumatoid arthritis in fingers and knees, lower back pain, knee and finger sports injuries, long-standing tendonitis, arthritic knees, heel pain as well as one subject who recently showed relief from greater occipital neuralgia (Arnold's neuralgia) and another subject who showed significant relief of pain from post-surgery back pain. Major symptom relief occurred within 1-10 days of treatment. Normally the subjects received the soy kefir powder of the present invention for a period of 2-3 weeks. Pain symptoms usually were fully re-established within several days of discontinuing the soy kefir powder of the invention. In cases of subjects who received the soy kefir powder of the present invention once again after discontinuance, substantial reductions in pain symptoms would re-occur.

### Open Label Efficacy Study

Evidence indicated that the soy kefir powder of the present invention may improve several features of the chronic fatigue syndrome, i.e., weakness, lack of energy and strength, pain, and depressed mood. Therefore, an open label pilot study was carried out to test the tolerance and effects of the product on a small group of patients, most of whom experienced chronic pain. Eleven patients received 56 pouches of 37.5 grams of product, to be taken as 1 pouch twice a day for 4 weeks. Patients answered the SF-36v2 (publicly available from http://www.sf-36.org/demos/SF-36v2.html) Health survey quality of life questionnaire before and after the 4 week treatment period.

The SF-36v2 Health Survey, is a highly validated, widely-used health status assessment tool that measures eight concepts: physical functioning (PF), role limitations due to physical health (RP), bodily pain (BP), general health perceptions (GH), vitality (VT), social functioning (SF), role limitations due to emotional problems (RE), and general mental health (MH). Scores for people at the top or bottom of a scale can be interpreted by looking at the items and response choices that must be chosen to earn those scores. For example, someone at the top score of the SF-36 Physical Functioning (PF) scale does not have limitations in any of the SF-36 activities due to health. A person scoring at the bottom of the PF scale is very limited in all activities, including bathing and dressing. Physical Functioning, Role Physical, and Bodily Pain are primarily measures of physical health, while the other three scales are primarily measures of mental health. Research has demonstrated that scales associated with the physical health construct are sensitive to detecting the impact of physical health interventions (Ware and Kosinski, 2001). Similarly, scales that are the strongest measures of mental health are sensitive in detecting the impact of mental health interventions. SF-36 is a FDA approved tool that is used in a wide variety of clinical areas ranging from cardiac rehabilitation programs and hip replacement surgery to the impact of medications on pain relief.

In the open label efficacy trial, two patients had to discontinue the treatment: one because of gastric pain after 3 days even though she said she had never felt so energetic from the time that she had the disease. She suggested that she wanted to try to take the product 1 or 2 days a week as her improvements were so remarkable. The second patient had gastric discomfort and vomited at her first ingestion of the product. Increased satiety effects were also noted after the ingestion of the product. All other patients took the product for 4 weeks. For the statistical analysis, a two-tailed Wilcoxon test was used. The results of the questionnaire are assembled into 8 scales and the average score for each scale before and after the treatment were compared. The alpha risk was 5%.

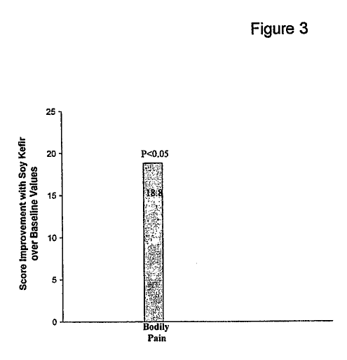

The results of this pilot study show that the product had significant beneficial effects on the subjects in terms of pain (FIG. 3). Bodily Pain showed differences with an alpha risk <5%. The clinical trial was extended from a two week to a four week intervention as there is evidence that the placebo effects typically fade after a two-week time frame. Hence, the placebo effect was therefore less likely as positive results were seen over the more extended period of four weeks.

Although only 3 of the 9 subjects showed spectacular effects such as the disappearance of a pain or more hours of functioning, there were still statistically significant effects that were described due to smaller improvements in these indices in the other patients. The results of the SF-36v2 Health Survey on pain relief showed an improvement of mean scores for bodily pain of 18.8 points with the soy kefir powder of the present invention, which compares favorably with other pain medications such as gabapentin used in the treatment of neuralgia (9.2 points) (Stacey et al., 2004) or a morphine sulfate drug (Kadian) used in an open-label trial for the treatment of moderate to severe nonmalignant pain (16 points) (Nicholson et al., 2003).

### Dosage and Administration

The dosage form is in dried powder provided in 17.5 or 35 gram sachets. The powder appears off-white to light tan in color and it can be dissolved in any liquid. To prepare the powder for consumption, a single sachet is mixed in at least 250 ml of fruit juice. The type of fruit juice is left to the discretion of the individual and, if the individual prefers, he/she may use a greater volume of juice. The formulated powder dissolves nicely into water and other liquids and has a pleasant, mild raspberry flavour.

### Summary

The soy kefir powder of the present invention has beneficial effects on chronic pain. Patients suffering from pain that are not adequately treated by conventional medicine, such as fibromyalgia may be a good population. Alternatively, or in addition, patients who suffer from chronic pain for whom treatment may be harmful, such as arthritis could be considered.

### Example 6

### Stability of the Soy Kefir Powder of the Invention

### Introduction

The Applicant reports results for stability conditions at 4° C., 20° C. and 30-32° C. with soy kefir powder of the present invention without added protectants. The addition of protectants consistently showed lowered content of bioactive components at all storage conditions as compared to the untreated soy kefir powder.

### Isoflavones

The three soybean isoflavone glycosides are genistin, daidzin, and glycitin, and their respective aglycones genistein, daidzein, and glycitein. Typically, more genistein/genistin exists in soybeans and soy foods than daidzein/daidzin whereas glycitein/glycitin comprises less than 10% of the total isoflavone content of soybeans (Murphy et al., 1999). Isoflavones are present mostly as β-glucoside conjugates which includes daidzin, genistin, and glycitin, and their malonyl and acetyl derivatives. The aglycones are the most bioactive isoflavones as they are absorbed faster and in higher amounts than their glucoside counterparts in vivo, which require extensive metabolism by the intestinal bacteria to yield the free aglycones. Typically, genistein occurs in higher concentrations than daidzein in soy foods. It is likely that aglycone isoflavones represent a major component of spray-dried soy kefir involved in pain modulation. Shir et al. (2002) have reported that moderate plasma concentrations of isoflavones have pain-suppressing properties in rats. Isoflavones have been postulated to inhibit pain via modulation of cytokines and antioxidant effects, thereby exerting anti-inflammatory action (Tall and Srinivasa, 2004).

The aglycone concentration of plain soymilk is typically less than 10%; however, fermentation of soymilk with probiotic bacteria such as Lactobacillus and Bifidobacterium sp. can hydrolyze the isoflavone glucosides into bioactive aglycones and increase the proportion of aglycones to approximately 50% of the total isoflavone content (Tsangalis et al., 2002, Otieno et al., 2006). Probiotic microorganisms possess β-glucosidase, β-galactosidase, and α-galactosidase, which play an important role in hydrolyzing isoflavone glucosides to bioavailable aglycones forms in fermented soymilk (Tochikura et al., 1986). Daidzein and genistein in soymilk have concentrations ranging from 1.90-4.45 mg/100 g (daidzein) and 2.81-6.06 mg/100 g (genistein) (USDA, 2001; United Soybean Board, 2001; Tsangalis et al., 2004; USDA, 2004). The normal range of the aglycone forms of isoflavones reported for non-fermented soymilk is significantly lower than the values in the kefir-fermented soy powder of the present invention at day 0 of storage as measured by KGK Synergize Inc. (London, ON, Canada). Thus, the aglycone isoflavone concentrations in soy kefir powder of the present invention soy kefir were 4.51 mg/35 g (diadzein) and 8.23 mg/35 g (genistein), which represent an approximate three-fold and four-fold increase in diadzein and genistein content, respectively, relative to the highest aglycone concentrations observed in unfermented soymilk. The diadzin and genistin content at day 0 of storage was 5.22 mg/35 g and 17.22 mg/35 g, which indicate that aglycones constitute approximately 57% of the total isoflavone content.

The change in isoflavone content at different storage conditions carried out in 2006 is shown in Table 15. Decreases were in total isoflavones were observed at higher temperature storage conditions of 30-32° C. and 20° C.

The relative values for 32° C. and 20° C. values over storage time seem relatively consistent. They show that glycoside isoflavones to be stable over time whereas drops in daidzein and genistein occurred with major decreases observed at 32° C. in genistein. In looking at the drop in the relative isoflavone values over time using the same operator, no apparent decrease occurred at 32° C. in terms of the glycoside isoflavones and minimal decreases were observed at 20° C. (i.e., 5% and 7% decreases for daidzin and genistin, respectively). Conversely, at 4° C. there was a 13% and 20% decreases in daidzin and genistin, respectively, which might be explainable by bacterial β-glucosidase activity noted to occur in stored fermented soymilk at 4° C., as discussed above.

Major decreases in the aglycone isoflavones, daidzein and genistein, with prolonged storage at 32° C. whereas the glycosidic isoflavones, diadzin and genistein, were relatively unchanged. At 20° C., there also appeared to be minimal changes in daidzin and genistin content; however, a major drop in daidzein occurred with a tendency for an increase in genistein. These results are consistent with previous literature indicating that there is minimal or no change in diadzin or genistin concentrations in soymilk during storage in the temperature range of 15-37° C. (Eisen et al., 2003). The specific degradation of daidzein as opposed to other isoflavones at 20° C. might be as a result of the greater lability of daidzein to thermal degradation (Ungar et al., 2003). The mechanisms by which isoflavones are lost during storage are unclear although some workers have indicated for formation of conjugates with simple sugars (Wang et al., 1990) or the formation of Maillard reaction products (Davies et al., 1998).

The isoflavone stability profile of fermented soymilk has not been tested previously to the knowledge of the Applicant as there is no published literature in this regard. It should be noted that in the previous literature, the fermented soymilk was inoculated with single individual bacteria strains with potent β-glucosidase activity as opposed to the kefir grain bacteria, which represent a mixture of bacteria, only some of which likely to have β-glucosidase activity. Other bacterial strains, however, with minimal β-glucosidase activity may generate other beneficial bioactive components. There is little or no change in diadzin and genistin concentrations in soymilk at 15-37° C. (Eisen B., Ungar Y., Shimoni E. 2003. Stability of isoflavones in soymilk stored at elevated and ambient temperatures. J. Agric. Food Chem., 51, 2212-2215), which parallels the lack of change in these two isoflavones as shown for soy kefir powder of the present invention at 20° C. and 32° C.

It should be noted that the sample used at 2 months storage was composed of 100% powder stuck to spray dryer walls as opposed to the powder of 63% extruded and 37% stuck used in all other samples for the stability study. Thus, the isoflavone values are very different for the 100% stuck powder used in the 2 month stability test, which makes this time point not a valid comparison versus the other time points at 4° C. and all the other temperature storage data, which all used powder consisting of a ratio of 63% extruded and 37% stuck powder.

The three fold higher daidzin concentration in the heat-exposed 100% powder stuck in the spray dryer (i.e., 2 month 4° C. values depicted in green versus 4° C. samples at stored at 1 month and 4 months that were composed of 63% extruded and 37% stuck powder) is explainable by an increase of 6-O-acetyldaidzin that occurs with higher heat exposure of soymilk, which subsequently deacetylates to form daidzin (Eisen B., Ungar Y., Shimoni E. 2003. Stability of isoflavones in soymilk stored at elevated and ambient temperatures. J. Agric. Food Chem., 51, 2212-2215). There were marked decreases in aglycones at 32° C., which was observed to a lesser extent at 20° C.; however, a major decrease in daidzein of 33% occurred at 20° C., which occurred to a lesser extent at 4° C. (19%).

In contrast to unchanged values of diadzin and genistin at higher storage temperatures, it appeared that these glycosidic isoflavones showed drops at 4° C. at 4 month storage. This latter observation may suggest that bacterial β-glucosidase activity during storage might be biotransforming isoflavones to the more bioactive aglycone forms. Recent work has indicated that β-glucosidase activity mediated by bacteria occurs during cold storage of fermented soymilk at 4° C., which enzymatically biotransforms isoflavone β-glucosides to their bioactive aglycone forms (Otieno et al., 2005). High levels of bacterial β-glucosidase activity for 5-6 weeks were maintained at cold storage (4° C. or −80° C.) with declines in activity occurring with more prolonged storage at 8 weeks (Otieno et al., 2005). Significantly higher levels of bacterial activity occurred at cold storage during all 8 weeks of cold storage as compared to storage at 24.8° C. or 37° C. (Otieno et al., 2005). Low temperatures apparently restrict cellular activity and metabolism to allow for better microbial cell stability and enzymatic activity. This latter study thus indicates that viable bacterial activity in liquid fermented soymilk can be maintained until at least 8 weeks of cold storage. Although the bacterial bioactivity of the spray-dried soy kefir has not been tested, microorganisms are less stable in fluid than dried products as they survive better at low-water activity (Wang et al., 2004). The isoflavone data suggests that β-glucosidase activity occurs in spray-dried soy kefir indicating the presence of bioactive probiotic microorganisms is maintained under cold storage at 4° C. In that regard, previous work has shown approximately 50-70% survival of lactic acid bacteria in spray-dried fermented soymilk stored at 4° C., which showed much higher percentage of bacterial survival as compared to storage of spray-dried fermented soymilk at 25° C. (Wang et al., 2004).

Another consideration is that that the major drop in total isoflavones at 4 months might also be associated with an increase in equol production from bacterial action during storage at 4° C. There is accumulating evidence that fermentation of daidzein to equol in the gut is responsible for a large part of the bioactivity of isoflavones as responders to soy have gut bacteria that generate equol. The health benefits among individuals to soy intake have been closely related to the ability of their gut bacteria to generate equol, i.e., high equol producers show much better health benefits. Equol is not normally present in soy foods and it was thought that only human gut bacteria can produce equol; however, equol has recently been shown to be formed in soymilk fermented by Bifidobacterium (Tsangalis et al., 2003). Although equol levels were not assessed, significant transformation of the isoflavones into this form might have occurred since there appears to be significant microbial activity at 4° C. and as fermentation of soymilk can lead to increase in equol. Hence, the apparent decrease in total isoflavones at 4° C. might also be due an increase in equol, the most bioactive and potent form of isoflavone.

As shown in Table 16, clinical efficacy of the soy kefir powder of the present invention against pain was maintained up to one year of storage at either ambient temperature or in cold storage conditions of 4° C.

Please see Table 17 that describes the isoflavone content for the older stored samples. It is noteworthy that the aglycone content of the November 2005 batch is 9.49 mg/35 g powder and the isoflavone content is still relatively high at approximately 57 mg/35 g powder despite prolonged storage at 4 C.

It is critical to note that there was less than optimal production of the soy kefir powder of the present invention for the 2006 stability study, i.e., due to lack of grains there was a diluted ratio of kefir grains to soymilk ranging from 1:60 to 1:90 versus previous established production protocol using 1:20 to 1:40. Also, powder that stuck to spray dryer walls (thereby exposed to high heat) was used in the stability study (a ratio of 63% extruded and 37% stuck powder) as opposed to 100% extruded powder used in the 2004 and 2005 production.

### Sphingomyelin

Sphingolipids are complex molecules composed of a sphingoid long-chain base of 14-22 carbon atoms (amino alcohol) having one amide-linked fatty-acyl chain and a polar head group (in contrast to the more common glycerolipids such as phospholipids or galactolipids that have two fatty-acyl chains and a polar head group linked to a glycerol backbone). There are over 300 different sphingolipids that have been characterized structurally, and when variations in the long-chain base and acyl chain are also considered, the possible molecular species number in the thousands (Vesper et al., 1999).

Animal food products and some plant food products (especially soybeans) contain substantial amounts of sphingolipids (Vesper et al., 1999). Soy has a single cerebroside, glucosylceramide, with d18:0, d18:1 D4, d18:1 D8, d18:2 D4,8, t18:0, t18:1 D8 and 16:0-26:0 fatty acids (including α-hydroxy and α,β-dihydroxy fatty acids) (Ohnishi and Fujino 1982). Sphingomyelin compounds were also noted by KGK Labs in the spray-dried soy kefir, which is normally found in high concentrations in bovine milk. The sphingomyelin species measured by KGK might be a kefir fermentation product of soymilk.

Dietary sphingomyelin is not absorbed intact but is metabolized to ceramide, phosphocholine, sphingosine, and fatty acids in the gut. There is evidence for an inhibitory effect on cholesterol absorption and antitumor effects, which may be mediated by the signaling effects of sphingomyelin metabolites (Nilsson and Duan, 2006). Central nervous system effects of dietary sphingomyelin are conceivable as dietary sphingomyelin contributes to CNS myelination in developing rats (Oshida et al., 2003). As shown in Table 18, there were decreases upon storage of the different species of sphingomyelin, with major drops observed particularly in the C16:16 species. There is no scientific literature support, however, for a role for these compounds in terms of pain regulation. Hence, the observed decrease in sphingomyelin content with storage is likely of lesser importance as compared to isoflavone content.

### Fatty Acids

The free fatty acid profiles shown in Tables 19 and 20 reflect those normally observed in soy lipids. There were minimal changes observed in the fatty acid profiles with storage. Minor fatty acid components, i.e., myristic, arachidic and behenic acids, which constituted <0.1% of total fatty acid content from our earlier fatty acid analysis in our lab, appeared to diminish with storage. None of these saturated free fatty acids, however, are associated with health benefits and thus the changes in these minor components are not likely significant. No major decreases in the major fatty acid components appear to occur under at either 4° C. or under accelerated storage conditions at 30-32° C.

### Peptides

The amino acid and peptide content of spray-dried soy kefir during storage at either 30-32° C. or 4° C. is shown in Tables 21 to 24. There were increases in both peptide and amino acid content at both storage conditions; however, these increases were more rapid at the higher storage temperature, which is likely due to thermal degradation of the peptides to smaller fragments and amino acids. There is an initial drop in amino acid content at 4° C. after 4 weeks of storage, which might be due to microbial consumption. The subsequent increase in amino acid content at 8 weeks of storage and the increases in peptide content that occur at 4° C. is probably due to microbial breakdown of peptides to more numerous smaller molecular weight peptides and amino acids. The peptides and amino acids were not identified by KGK lobs.

### Conclusions

According to the data mentioned in this example, the stability of soy kefir powder of the present invention at 4° C. appears to the most appropriate condition according to the aglycone content. The apparent drop in isoflavone content is consistent with maintenance of bacterial activity leading to greater production of the more bioactive aglycones, which could lead to an increased isoflavone bioactivity upon storage at 4° C. for the initial few months of storage. The content of free fatty acids was not greatly affected by storage. The changes observed in amino acid and peptide content with storage is difficult to interpret since this could either involve the loss of important amino acids or peptides or conversely lead to the generation of more bioactive amino acid and peptides species with prolonged storage. The sphingomyelin content was depressed upon storage at higher temperatures indicating that these components are relatively unstable although they have not been linked with pain modulatory effects such as isoflavones. There are several other bioactive components that likely work together with isoflavones in modulating pain which were not measured. In addition to isoflavones, soy kefir likely contains a host of components that may be involved in the pain relieving effects including polyphenolic compounds such as lignans, saponins, phytic acid and other phytochemicals, which might exert cumulative biological effects on pain. For example, saponins and polyphenolic compounds, which are found in rich concentrations in soy, have been suggested to exert anti-arthritic effects (Cheeke et al., 2006). The observations of concerning clinical efficacy of >1 year old soy kefir powder of the present invention provides some initial assurance that the key soy kefir components remain relatively stable at least in terms of some clinical parameters.

The stability of the product is optimal at 4° C. in a non-formulated form as indicated by the stability study and as the 2005 samples appear to have with a shelf life of one year in terms of their clinical efficacy. It is noteworthy that despite changes in peptide, and sphingomyelin profiles, major pain relieving effects were observed indicating either that these components did not play a significant role or that the changes in their profiles were not modulated sufficiently during storage to alter significantly pain relieving effects. Using the isoflavone content of the 2005 powder as the basis of the specification data, this would correspond to 37 mg as the minimal total isoflavone content and a minimal content of 8 mg aglycones as the 2005 product has been associated with clinical efficacy over a period of approximately one year. In conclusion, we indicate a specification of shelf-life of one year at 4° C. with the above isoflavone content for pain modulatory effects.

TABLE 1
Effect of prior art treatment and the soy kefir powder of the present
invention on blood pressure

| | | | Change | Change | |
|---|---|---|---|---|---|
| | | Duration | DBP | SBP | |
| Treatment | Dose | weeks | mmHg | mmHg | Reference |
| Tryptic casein | 20 g/day | 4 | −4.6 | −6.6 | 111 |
| Calpis | 95 mL/day | 8 | −6.9 | −14.1 | 112 |
| Lactobacillus | 150 mL/day | 8 | −8.8 | −14.9 | 113 |
| helveticus LBK- | | | | | |
| 16 H Fermented | | | | | |
| cow's milk | | | | | |
| Lactobacillus | 150 mL/day | 21 | −3.6 | −6.7 | 114 |
| helveticus LBK- | | | | | |
| 16 H Fermented | | | | | |
| cow's milk | | | | | |
| C12 | >0.2 g/day | 4 | −6.5 | −4.5 | 115 |
| Biozate whey | 20 g/day | 6 | −7.0 | −11.0 | 116 |
| peptides | | | | | |
| Fermented cow's | 100 mL/day | 12 | −7.2 | −17.4 | 117 |
| milk product | | | | | |
| containing GABA | | | | | |
| using two kinds of | | | | | |
| starters - | | | | | |
| Lactobacillus | | | | | |
| casei strain | | | | | |
| Shirota, and | | | | | |
| Lactococcus | | | | | |
| lactis YIT 2027 | | | | | |
| Soy protein | 40 g of | 4 | No | No | 99 |
| containing | isolated soy | | Significant | Significant | |
| isoflavones | protein powder | | Change | Change | |
| | containing 80 mg | | | | |
| | isoflavones | | | | |
| | (60% | | | | |
| | genistein, 30% | | | | |
| | daidzein, 10% | | | | |
| | glycitein) per | | | | |
| | day | | | | |
| Isoflavone | 55 mg of | 8 | No | No | 100 |
| Supplement | isoflavonoids/day, | | Significant | Significant | |
| | including | | Change | Change | |
| | 30 mg of | | | | |
| | genistein, 16 mg | | | | |
| | of | | | | |
| | biochanin A, 1 mg | | | | |
| | of | | | | |
| | daidzein, and | | | | |
| | 8 mg of | | | | |
| | formononetin | | | | |
| Isoflavone | Genistein (54 mg/ | 24 | No | No | 101 |
| Supplement | day) | | Significant | Significant | |
| | | | Change | Change | |
| Isoflavone | 40 mg of total | 8 | No | No | 102 |
| Supplement | isoflavones/day | | Significant | Significant | |
| | | | Change | Change | |
| Isoflavone | 43.5 mg | 52 | No | No | 103 |
| Supplement | isoflavones/day | | Significant | Significant | |
| | | | Change | Change | |
| Soy protein | 25.6 g | 52 | +1.99 | +4.34 | 104 |
| containing | isoflavone-rich | | | | |
| isoflavones | soy protein | | | | |
| | containing 99 mg | | | | |
| | isoflavones/day | | | | |
| | (i.e., 52 mg | | | | |
| | genistein, 41 mg | | | | |
| | daidzein, | | | | |
| | and 6 mg | | | | |
| | glycitein) in | | | | |
| | 36.5 g soy- | | | | |
| | protein powder | | | | |
| Soy protein | 40 g soy | 12 | −4.3 | −7.5 | 118 |
| isolate containing | protein, 118 mg | | | | |
| isoflavones | isoflavones/day | | | | |
| Soybean milk and | 30 g soy | 5 | No | No | 105 |
| soybean yogurt | protein, 80 mg | | Significant | Significant | |
| | isoflavones/day | | Change | Change | |
| Soy milk | 500 mL twice | 12 | −18.4 | −15.9 | 119 |
| | daily | | | | |
| Low calorie soy- | | 12 | No | No | 106 |
| based meal | | | Significant | Significant | |
| replacement | | | Change | Change | |
| Soy protein | 20 g of soy | 6 | −5 | No | 107 |
| isolate containing | protein | | | Significant | |
| isoflavones | containing 34 mg | | | Change | |
| | of | | | | |
| | phytoestrogens | | | | |
| | split into two | | | | |
| | doses/day | | | | |
| Soy protein and | Diets | 5 | −5.1 | −10.8 | 120 |
| isoflavones | containing at least | | | | |
| supplement | 20 g Soy | | | | |
| | protein and atleast | | | | |
| | 80 mg | | | | |
| | isoflavones | | | | |
| High- and low- | High - (50 g | 4 | −4.0 | No | 108 |
| isoflavone | soy protein | | | Significant | |
| soyfood diets | and 73 mg | | | Change | |
| | isoflavones | | | | |
| | daily) and low - | | | | |
| | (52 g soy | | | | |
| | protein and 10 mg | | | | |
| | isoflavones | | | | |
| | daily) | | | | |
| | isoflavone | | | | |
| | soyfood | | | | |
| Fermented soy | | 6 | ¹6.4 | 0.0 | 109 |
| products | | | | | |
| (low-sodium soy | | | | | |
| sauce and miso) | | | | | |
| the soy kefir | 25 or 35 g/day | 4 | −6.3 | −17.3 | — |
| powder of the | | 8 | −5.5 | −25.5 | |
| invention | | | | | |

¹No significant change in BP was observed in the entire cohort; however, in those aged 40 years and older, a mild reduction in DBP was observed.

TABLE 2
Composition of the Microflora of the Kefir Grains from the All-Russian
Scientific Research Institute of Dairy Industry (ARDI)

| Microorganism | CFU/g | | Species | Total Sum | |
|---|---|---|---|---|---|
| Lactobacillus acidophilus* | 2.65 × 10⁸ | (87.78)^{a} | Lactobacilli | 2.92 × 10⁸ | (96.82) |
| Lactobacillus delbrueckii | 1.96 × 10⁶ | (0.65) | Lactococci | 5.12 × 10⁶ | (1.64) |
| lactis | | | | | |
| Lactobacillus kefiri (formerly | 2.5 × 10⁷ | (8.28) | Yeasts | 4.78 × 10⁶ | (1.53) |
| L. brevis) | | | | | |
| Lactobacillus kefiranofaciens | 4.00 × 10⁴ | (0.01) | Total | 3.02 × 10⁸ | |
| Leuconostoc mesenteroides | 1.80 × 10⁵ | (0.06) | | | |
| cermoris | | | | | |
| Lactococcus lactis lactis | 1.7 × 10⁶ | (0.91) | | | |
| Lactococcus lactis cremoris | 1.98 × 10⁶ | (0.66) | | | |
| Leuconostoc mesenteroides | 2.00 × 10⁵ | (0.07) | | | |
| mesenteroides | | | | | |
| Candida kefyr | 2.1 × 10⁶ | (0.70) | | | |
| Candida tenuis | 2.4 × 10⁵ | (0.08) | | | |
| Saccharomyces lactis | 1.26 × 10⁶ | (0.42) | | | |
| Saccharomyces unisporus | 1.18 × 10⁶ | (0.39) | | | |
| (Saccharomyces delbrueckii) | | | | | |

^{a}Data in parenthesis represent percentage of total microflora

TABLE 3a
Composition of the soy kefir powder of the present invention

| CONTROLS | SPECIFICATION | RESULTS | METHOD |
|---|---|---|---|
| Description | Fine Powder | Conform | Visual |
| Colour | Beige Powder | Conform | Visual |
| Odour | Slightly milk | Conform | Olfactive |
| pH | 4.0-4.5 | 4.22 | USP <791> |
| Protein | 29.4-44.2% | 36.8% | NPPF - 143T |
| Fat | 28.6-42.8% | 35.7% | NPPF - 083T |
| Carbohydrate | 9.6-14.4% | 12.0% | NPPF - 083T |
| Loss on drying | 7.6-11.4% | 9.5% | USP <731> |
| Ash | 4.8-7.2% | 6.0% | USP <281> |
| Total Plate Count | — | 5.7 × 10⁶ | USP <61> |
| | | org/g | |
| Yeast & Molds | <1000 cfu/g | 50 org/g | USP <61> |
| Salmonella spp. | Absent | Absent | USP <61> |
| S. aureus | Absent | Absent | USP <61> |
| E. Coli | Absent | Absent | USP <61> |
| P. aeruginosa | Absent | Absent | USP <61> |
| Enterobacter spp. | <100 cfu/g | Absent | MCB - 240802 |
| IC₅₀ | 2.6-4.0 mg/mL | 3.3 | OPA-Chromogenic |
| | | mg/mL | Reaction |

TABLE 3b
Isoflavone content of soy kefir powder of the present invention

| | Batch | | | | |
|---|---|---|---|---|---|
| | | | 2005 | | |
| | | | (pre- | 2005 Can | |
| | 2004 | 2005 | evaporated) | Met | 2006 |
| Daidzein μg/g | 88.37 | 81.67 | 46.76 | 53.93 | 102.66 |
| Daidzin μg/g | 1042.46 | 981.17 | 792.57 | 790.72 | 347.01 |
| Genistein μg/g | 183.96 | 170.93 | 75.72 | 85.06 | 174.24 |
| Genistin μg/g | 1201.63 | 1127.93 | 1412.72 | 1399.45 | 598.39 |
| Total Isoflavones | 2516.4 | 2361.7 | 2327.8 | 2329.2 | 1222.3 |
| μg/g | | | | | |
| % aglycone from | 10.8 | 10.7 | 5.3 | 6.0 | 22.7 |
| total isoflavone | | | | | |

TABLE 4
Results from a study of the effect of the soy kefir powder of the present invention on blood pressure

| Therapeutic | | Treatment | DBP/SBP | | Observations |
|---|---|---|---|---|---|
| Goal(s) | Dose | Duration | Baseline | End Treatment | (therapeutic outcomes, adverse events, etc.) |

Subject TK (77, M)

| | | | | | |
|---|---|---|---|---|---|
| BP reduction | 200 ml QD | Liquid (approx. | 87/164 | 77/129 | Subject had been receiving diuretic therapy for |
| Joint pain relief | (liquid) | 1 yr) | (Trial 1) | (powder - Trial 1) | elevated BP. Subject then received concomitant |
| | 25 g QD | 25 g Powder | 82/162 | 69/143 | therapy with liquid soy kefir for approximately 1 |
| | (powder) | (2 mo - Trial 1) | (Trial 2) | (powder - Trial 2) | year with an additional mild reduction of BP being |
| | 35 g QD | 35 g Powder | | | noted. Subject then switched to powder soy kefir |
| | (powder) | (approx 1 wk - | | | and experienced a further reduction in BP within 1 |
| | | Trial 2) | | | week (Trial 1). Diuretic was discontinued and |
| | | | | | lowered BP was maintained with only soy kefir |
| | | | | | powder. A similar reduction in BP was observed |
| | | | | | within one week using the soy kefir powder in Trial |
| | | | | | 2. |
| | | | | | Subject had 1-year history of chronic back pain |
| | | | | | prior to receiving soy kefir. Within 3 weeks of |
| | | | | | receiving liquid soy kefir, subject reported cessation |
| | | | | | of back pain. |
| | | | | | Subject received cow's milk kefir on a daily basis |
| | | | | | of for several months prior to receiving soy kefir |
| | | | | | with no observations of BP lowering or back pain |
| | | | | | relief. |
| | | | | | No adverse events were noted while receiving soy |
| | | | | | kefir (liquid or powder). |

Subject WK (72 F)

| | | | | | |
|---|---|---|---|---|---|
| BP | 200 ml | Liquid (approx | 88/168 | 87/152 | Subject had been receiving Hyzaar therapy for elevated BP. |
| reduction | QD | 1 yr) | (Trial 1) | (powder - | Subject then received concomitant therapy with liquid soy kefir for |
| Anti- | (liquid) | 25 g Powder | 92/172 | Trial 1) | approximately 1 year with an additional mild reduction of BP being |
| inflammation | 25 g QD | (2 mo - Trial 1) | (Trial 2) | 82/161 | noted. Subject then switched to powder soy kefir and experienced a |
| joint pain | (powder) | 35 g Powder | | (powder - | further reduction in BP within 1 week. |
| relief | | (approx 1 mo - | | Trial 2) | Throughout treatment, subject continued to receive thyroid |
| | | Trial 2) | | | replacement medication (Synthroid). |
| | | | | | Subject had history of inflammation and arthritic joint pain in |
| | | | | | fingers and knee. Within 3 weeks of receiving liquid soy kefir, |
| | | | | | Subject reported decreases in both inflammation and joint pain; |
| | | | | | these symptoms improved further upon switching to the soy kefir |
| | | | | | powder (Trial 1). |
| | | | | | Subject received cow's milk kefir for several months on a daily |
| | | | | | basis prior to receiving soy kefir with no observations of BP |
| | | | | | lowering or joint pain relief. |
| | | | | | No adverse events were noted while receiving soy kefir (liquid or |
| | | | | | powder). |

Subject PF (60, F)

| | | | | | |
|---|---|---|---|---|---|
| BP | 35 g | 35 g QD | 100/160 | 90/140 | Initial daily intake of 35 g QD over a period of 10 days showed no |
| reduction | QD | (10 d) | | | change in BP. |
| | (powder) | | | | BP reduction was noted following the next 10 days of 35 g BID |
| | | | | | soy kefir intake. BP elevation occurred to previously observed |
| | | | | | high levels subsequent to discontinuation of soy kefir. |
| | | | | | No adverse events noted. |

Subject SK (47, M)

| | | | | | |
|---|---|---|---|---|---|
| Anti- | 200 ml | Liquid | 79/130 | 75/121 | Daily serial BP measurements of alternating periods of with and |
| inflammation | QD | (approx 18 mo) | | (liquid) | without intake of soy kefir demonstrated BP lowering with liquid |
| Joint pain relief | (liquid) | 25 g Powder | | | soy kefir within 2 weeks. |
| BP reduction | 25 g QD | (2 wk - Trial 1) | | | Symptoms of pain due to knee injury (requiring brace during |
| | (powder) | | | | soccer play) increased in severity in pain with time. Symptom |
| | | | | | relief and decreased swelling in knee area was observed within 3 |
| | | | | | weeks of liquid soy kefir intake. Complete alleviation of pain |
| | | | | | within approximately 1 month. |
| | | | | | No adverse events were noted while receiving soy kefir (liquid or |
| | | | | | powder). |

Subject DL (56, F)

| | | | | | |
|---|---|---|---|---|---|
| Joint pain | 200 ml | Liquid | 53/96 | 50/96 | Chronic shoulder pain (bursitis) significantly alleviated within 2 |
| relief | QD | (2 mo) | | | weeks of liquid soy kefir intake. |
| | (liquid) | 35 g powder | | | Intake of the soy kefir powder (Trial 2) was associated with an |
| | 35 g QD | (approx 2 wk - | | | improvement in ratings related to joint pain, with baseline ratings |
| | (powder) | Trial 2) | | | of 4 for long-standing pain in both shoulder pain (bursitis) and |
| | | | | | neck pain, which improved to ratings of 1 (bursitis) and 2 (neck |
| | | | | | pain) within 4-5 days of initiation of daily the soy kefir powder |
| | | | | | intake. |
| | | | | | Subject had history of hypotension; no drop in BP was |
| | | | | | noted with the intake of soy kefir. |
| | | | | | No adverse events were noted while receiving liquid soy kefir. |

Subject RV (38, F)

| | | | | | |
|---|---|---|---|---|---|
| Joint pain | 200 ml | Liquid | No data | No data | Relief of chronic shoulder pain due to fall was observed within 3 |
| relief | QD | (4 wk) | | | days of intake of 350 ml QD liquid. Symptoms re-appeared within |
| | (liquid) | 35 g Powder | | | several days of discontinuing soy kefir. Symptom reduction was |
| | 350 ml | (approx 2 wk - | | | not noted with 200 ml QD. |
| | QD | Trail 2) | | | With intake of clinical batch of the soy kefir powder (Trial 2), |
| | (liquid) | | | | subject showed dramatic improvement of chronic pain related to |
| | 35 g QD | | | | long-standing tendonitis and a coccyx injury. Joint pain ratings |
| | (powder) | | | | improved from 5 to 2 within 5 days of intake of the soy kefir |
| | | | | | powder and further diminished to a rating of 0 within 2 weeks |
| | | | | | of intake. |
| | | | | | No adverse events were noted while receiving liquid |
| | | | | | soy kefir. |

Subject DV (44, F)

| | | | | | |
|---|---|---|---|---|---|
| Joint pain | 35 g QD | Powder | 77/122 | 73/117 | Arthritic hip caused acute pain during walking. Significant |
| relief | (powder) | (2 wk - Trial | | | alleviation of hip joint pain (walking without pain) noted within |
| | 35 g | 1) | | | three days of the soykefir powder consumption. |
| | Powder | 35 g Powder | | | Pain symptoms fully re-established within several days of |
| | (Trial 2) | (approx 2 wk - | | | discontinuing the soy kefir powder. Two separate episodes |
| | | Trial 2) | | | separated by a period of weeks were recorded of significant pain |
| | | | | | relief following intake of the soy kefir powder followed by |
| | | | | | recurrence of symptoms within days of discontinuance of the |
| | | | | | soy kefir powder. |
| | | | | | With intake of clinical batch of the soy kefir powder (Trial 2), |
| | | | | | subject showed improvement in ratings related to hip joint pain |
| | | | | | going from 4-5 to 1-2 within 2 weeks, which further diminished to |
| | | | | | 0-1 within 22 days. Fatigue ratings went from 3-4 to 0 within 15 |
| | | | | | days and stress/depression also went from ratings of 4-5 to 0 |
| | | | | | within 15 days. |
| | | | | | No adverse events were noted apart from a strong odor of |
| | | | | | flatulence in the first 10 days of intake of the soy kefir powder; |
| | | | | | no meaningful drop in BP was noted with the intake of the soy |
| | | | | | kefir powder. |

Subject PW (46, M)

| | | | | | |
|---|---|---|---|---|---|
| Joint pain | 350 ml | Liquid | No data | No data | Chronic arthritic knee pain caused difficulty during soccer play and |
| relief | QD | (approx 4 wk) | | | in basic ambulation (rising from bed, exiting car). |
| | (liquid) | 35 g | | | Within 1 week of soy kefir (liquid and powder) intake, significant |
| | 35 g QD | powder | | | alleviation in knee pain was noted; no pain observed while getting |
| | (powder) | (approx 4 wk - | | | out of bed/car or playing soccer. Pain relief augmented with |
| | | Trial 1) | | | increasing duration of therapy. Powder soy kefir appeared more |
| | | | | | efficacious than liquid. Cessation of intake led to gradual |
| | | | | | re-appearance of pain symptoms. |
| | | | | | No adverse events were noted while receiving soy kefir (liquid or |
| | | | | | powder). |

Subject LC (42, M)

| | | | | | |
|---|---|---|---|---|---|
| Heel pain | 200 ml | Liquid | No data | No data | Significant alleviation of chronic heel pain noted with intake of |
| relief | QD | (3 wk) | | | liquid soy kefir. Pain recurred upon of cessation of soy kefir. |
| | (liquid) | | | | No adverse events were noted while receiving liquid soy kefir. |

Subject MM (50, F)

| | | | | | |
|---|---|---|---|---|---|
| None | 200 ml | Liquid | 57/90 | 57/90 | No adverse events were noted while receiving liquid soy kefir; no |
| | QD | (10 days) | | | drop in BP was noted with the intake of soy kefir. |
| | (liquid) | | | | |

Subject KP (71, F)

| | | | | | |
|---|---|---|---|---|---|
| BP | 35 g QD | 35 g Powder | 76/162 | 73/151 | Subject took the soy kefir powder (Trial 2) on an irregular basis |
| reduction | (powder) | (approx 4 wk - | | | approximately once every two days. |
| | | Trial 2) | | | No adverse events were noted while receiving the soy kefir |
| | | | | | powder. |

Subject AC (F, 52)

| | | | | | |
|---|---|---|---|---|---|
| Anti- | 200 ml QD | Liquid | 67/104 | 63/111 | Subject had previously consumed cow's milk kefir daily for |
| inflammation | (liquid) | (approx 18 mo) | | | several months without any apparent symptom relief. |
| Joint pain | 25 g QD | Powder | | | Chronic knee pain that interfered with jogging disappeared |
| relief | (powder) | (approx 2 wk - | | | following intake of soy kefir. |
| | | Trial 1) | | | No adverse events were noted while receiving soy kefir (liquid or |
| | | | | | powder). |

Subject JK (14, F)

| | | | | | |
|---|---|---|---|---|---|
| Pain relief | 35 g QD | 35 g Powder | No data | No data | Subject experienced moderate to severe chronic back pain for two |
| | (powder) | (approx 2 wk - | | | months following surgery for scoliosis for which she was taking |
| | | Trial 2) | | | daily 8-10 pills of acetaminophen and/or a narcotic analgesic |
| | | | | | (Dilaudid). Within one day of intake of clinical batch of the soy |
| | | | | | kefir powder (Trial 2), subject showed major improvement in |
| | | | | | ratings related to pain going from 4.5 to 0. Sever pain symptoms |
| | | | | | re-appeared after 48 hours following withdrawal of the soy kefir |
| | | | | | powder. Pain symptoms disappeared once again within 24 |
| | | | | | hours of intake of the soy kefir powder. |
| | | | | | No adverse events were noted while receiving while receiving the |
| | | | | | soy kefir powder. |

TABLE 5
Results from a study of the effect of the soy kefir
powder of the present invention on pain relief

| SUBJECT | DOSE | DURATION | CHANGE IN PAIN SYMPTOM RATING¹ |
|---|---|---|---|
| Subject DK | 35 g QD | 2 weeks | Subject suffered from chronic pain due to |
| (46, F) | (powder) | | ankylosing spondylitis. |
| | | | Joint pain ratings improved from 4-5 to 2.5-3. |
| Subject DL | 35 g QD | 2 weeks | Intake of the soy kefir powder was associated |
| (56, F) | (powder) | | with an improvement in ratings related to joint |
| | | | pain, with baseline ratings of 4 for long-standing |
| | | | pain in both shoulder pain (bursitis) and neck pain, |
| | | | which improved to ratings of 1 (bursitis) and 2 |
| | | | (neck pain) within 4-5 days of initiation of daily the |
| | | | soy kefir powder intake. |
| Subject RV | 35 g QD | 2 weeks | With intake of clinical batch of the soy kefir |
| (38, F) | (powder) | | powder, subject showed dramatic improvement |
| | | | of chronic pain related to long-standing tendonitis |
| | | | and a coccyx injury. |
| | | | Joint pain ratings improved from 5 to 2 within 5 |
| | | | days of intake of the soy kefir powder and further |
| | | | diminished to a rating of 0 within 2 weeks of |
| | | | intake. |
| Subject DV | 35 g QD | 4 weeks | Subject had chronic long term arthritic hip pain, |
| (44, F) | (powder) | | which also caused acute pain during walking. With |
| | | | intake of clinical batch of the soy kefir powder, |
| | | | subject showed improvement in ratings related to |
| | | | arthritic hip joint pain going from 4-5 to 1-2 within |
| | | | 2 weeks, which further diminished to 0-1 within 22 |
| | | | days. |
| | | | Pain symptoms fully re-established within several |
| | | | days of discontinuing soy kefir powder. Two |
| | | | separate episodes separated by a period of weeks |
| | | | were recorded of significant pain relief following |
| | | | intake of soy kefir powder followed by recurrence |
| | | | of symptoms within days of discontinuance of soy |
| | | | kefir powder. |
| Subject JK | 35 g QD | 3 weeks | Subject experienced moderate to severe chronic |
| (14, F) | (powder) | | back pain for two months following surgery for |
| | | | scoliosis for which she was taking daily 8-10 pills |
| | | | of acetaminophen and/or a narcotic analgesic |
| | | | (Dilaudid). Within one day of intake of clinical |
| | | | batch of soy kefir powder (Trial 2), subject |
| | | | showed major improvement in ratings related to |
| | | | pain going from 4.5 to 0. |
| | | | Severe pain symptoms re-appeared after 48 hours |
| | | | following withdrawal of soy kefir powder. Pain |
| | | | symptoms disappeared once again within 24 |
| | | | hours of intake of soy kefir powder. |

¹Symptoms regarding joint pain were rated on a scale 0-5 with a 0 being symptom free and 5 meaning symptom at its greatest intensity.

TABLE 6
Adverse effects with the intake of soy kefir powder of the present
invention

| | According to treatment group | | | | |
|---|---|---|---|---|---|
| | Soy Kefir | | Placebo | | p- |
| | Patients | Events | Patients | Events | value¹ |
| N | 21 | | 20 | | |
| AEs | 14 (66.7%) | 26 | 9 (45.0%) | 13 | 0.2059 |
| SAEs | 0 (0.0%) | 0 (0.0%) | 0 (0.0%) | 0 (0.0%) | — |
| AEs related to study | 2 (9.5%) | 2 (7.7%) | 0 (0.0%) | 0 (0.0%) | — |
| treatment² | | | | | |
| AEs possibly related to | 11 (52.4%) | 16 (61.5%) | 5 (25.0%) | 7 (53.8%) | 0.2059 |
| study treatment³ | | | | | |

¹Friedman's test
²Probable or definite relationship to treatment
³Possible, probable or definite relationship to treatment

TABLE 7
Effects of the intake of soy kefir powder of the present invention on ALT

| | | Soy Kefir | | Placebo | |
|---|---|---|---|---|---|
| ALT (U/L) | | (n = 23) | | (n = 23) | |
| Baseline | N | 23 | | 23 | |
| | Mean ± SD | 24.8 ± 10.5 | | 24.8 ± 10.5 | |
| | Median (P25, P75) | 21.0 | (17.0, 30.0) | 21.0 | (17.0, 30.0) |
| | Min, Max | 10.0, 52.0 | | 10.0, 52.0 | |
| | Normal | 23 | (100.0%) | 23 | (100.0%) |
| End of Treatment | N | 18 | | 20 | |
| | Mean ± SD | 24.7 ± 11.2 | | 23.7 ± 10.7 | |
| | Median (P25, P75) | 21.5 | (19.0, 27.0) | 23.0 | (15.5, 27.5) |
| | Min, Max | 11.0, 58.0 | | 9.0, 54.0 | |
| | Normal | 17 | (94.4%) | 19 | (95.0%) |
| | High (not clinically significant) | 1 | (5.6%) | 1 | (5.0%) |
| | Missing | 5 | | 3 | |
| Change from Baseline | N | 18 | | 20 | |
| | Mean ± SD | −0.94 ± 5.06 | | −2.05 ± 4.50 | |
| | Median (P25, P75) | −1.00 | (−4.00, 2.00) | −1.50 | (−5.50, 1.50) |
| | Min, Max | −10.00, 10.00 | | −9.00, 7.00 | |
| | P-value (within-group) | 0.4392 | (T-Test) | 0.0559 | (T-Test) |

TABLE 8
Effects of the intake of soy kefir powder of the present invention on AST

| | | Soy Kefir | | Placebo | |
|---|---|---|---|---|---|
| AST (U/L) | | (n = 23) | | (n = 23) | |
| Baseline | N | 23 | | 23 | |
| | Mean ± SD | 22.4 ± 5.1 | | 22.4 ± 5.1 | |
| | Median (P25, P75) | 21.0 | (19.0, 26.0) | 21.0 | (19.0, 26.0) |
| | Min, Max | 16.0, 38.0 | | 16.0, 38.0 | |
| | Normal | 23 | (100.0%) | 23 | (100.0%) |
| End of Treatment | N | 18 | | 20 | |
| | Mean ± SD | 23.0 ± 7.5 | | 22.0 ± 5.9 | |
| | Median (P25, P75) | 21.0 | (19.0, 24.0) | 22.5 | (17.0, 25.0) |
| | Min, Max | 15.0, 42.0 | | 14.0, 40.0 | |
| | Normal | 17 | (94.4%) | 20 | (100.0%) |
| | High (not clinically significant) | 1 | (5.6%) | 0 | (0.0%) |
| | Missing | 5 | | 3 | |
| Change from Baseline | N | 18 | | 20 | |
| | Mean ± SD | 0.44 ± 6.16 | | −0.65 ± 3.15 | |
| | Median (P25, P75) | −1.00 | (−2.00, 1.00) | −1.00 | (−2.00, 1.00) |
| | Min, Max | −5.00, 23.00 | | −8.00, 7.00 | |
| | P-value (within-group) | 0.3988 | (Signed Rank) | 0.3677 | (T-Test) |

TABLE 9
Effects of the intake of soy kefir powder of the present invention on total bilirubin

| | | Soy Kefir | | Placebo | |
|---|---|---|---|---|---|
| Bilirubin Total (μmol/L) | | (n = 23) | | (n = 23) | |
| Baseline | N | 23 | | 23 | |
| | Mean ± SD | 13.7 ± 6.2 | | 13.7 ± 6.2 | |
| | Median (P25, P75) | 12.0 | (9.0, 17.0) | 12.0 | (9.0, 17.0) |
| | Min, Max | 8.0, 35.0 | | 8.0, 35.0 | |
| | Normal | 22 | (95.7%) | 22 | (95.7%) |
| | High (not clinically significant) | 1 | (4.3%) | 1 | (4.3%) |
| End of Treatment | N | 18 | | 20 | |
| | Mean ± SD | 14.6 ± 5.5 | | 12.7 ± 6.4 | |
| | Median (P25, P75) | 12.5 | (10.0, 16.0) | 12.0 | (9.0, 14.0) |
| | Min. Max | 9.0, 29.0 | | 6.0, 35.0 | |
| | Normal | 17 | (94.4%) | 19 | (95.0%) |
| | High (not clinically significant) | 1 | (5.6%) | 1 | (5.0%) |
| | Missing | 5 | | 3 | |
| Change from Baseline | N | 18 | | 20 | |
| | Mean ± SD | 0.11 ± 5.79 | | −1.25 ± 3.39 | |
| | Median (P25, P75) | 1.00 | (−4.00, 3.00) | −1.00 | (−4.00, 1.00) |
| | Min, Max | −10.00, 12.00 | | −9.00, 4.00 | |
| | P-value (within-group) | 0.9361 | (T-Test) | 0.1151 | (T-Test) |

TABLE 10
Effects of the intake of soy kefir powder of the present invention on CK

| | | Soy Kefir | | Placebo | |
|---|---|---|---|---|---|
| CK (U/L) | | (n = 23) | | (n = 23) | |
| Baseline | N | 23 | | 23 | |
| | Mean ± SD | 99.1 ± 45.9 | | 99.1 ± 45.9 | |
| | Median (P25, P75) | 87.0 | (60.0, 123.0) | 87.0 | (60.0, 123.0) |
| | Min, Max | 40.0, 203.0 | | 40.0, 203.0 | |
| | Low (not clinically significant) | 1 | (4.3%) | 1 | (4.3%) |
| | Normal | 21 | (91.3%) | 21 | (91.3%) |
| | High (not clinically significant) | 1 | (4.3%) | 1 | (4.3%) |
| End of Treatment | N | 18 | | 20 | |
| | Mean ± SD | 99.7 ± 40.2 | | 97.2 ± 40.4 | |
| | Median (P25, P75) | 94.0 | (66.0, 135.0) | 94.5 | (68.0. 119.0) |
| | Min, Max | 38.0, 171.0 | | 33.0, 185.0 | |
| | Low (not clinically significant) | 1 | (5.6%) | 2 | (10.0%) |
| | Normal | 17 | (94.4%) | 18 | (90.0%) |
| | Missing | 5 | | 3 | |
| Change from Baseline | N | 18 | | 20 | |
| | Mean ± SD | −0.33 ± 23.53 | | −5.00 ± 38.54 | |
| | Median (P25, P75) | 3.00 | (−19.00, 11.00) | −5.50 | (−33.00, 9.00) |
| | Min, Max | −44.00, 42.00 | | −80.00, 83.00 | |
| | P-value (within-qroup) | 0.9528 | (T-Test) | 0.5686 | (T-Test) |

TABLE 11
Effects of the intake of soy kefir powder of the present invention on creatinine

| | | Soy Kefir | | Placebo | |
|---|---|---|---|---|---|
| Creatinine (μmol/L) | | (n = 23) | | (n = 23) | |
| Baseline | N | 23 | | 23 | |
| | Mean ± SD | 82.7 ± 14.1 | | 82.7 ± 14.1 | |
| | Median (P25, P75) | 81.0 | (72.0, 91.0) | 81.0 | (72.0, 91.0) |
| | Min, Max | 50.0, 117.0 | | 50.0, 117.0 | |
| | Low (not clinically significant) | 1 | (4.3%) | 1 | (4.3%) |
| | Normal | 22 | (95.7%) | 22 | (95.7%) |
| End of Treatment | N | 18 | | 20 | |
| | Mean ± SD | 87.1 ± 14.4 | | 84.2 ± 12.6 | |
| | Median (P25, P75) | 87.5 | (77.0, 101.0) | 84.5 | (77.0, 93.5) |
| | Min, Max | 57.0, 109.0 | | 50.0, 103.0 | |
| | Low (not clinically significant) | 1 | (5.6%) | 1 | (5.0%) |
| | Normal | 17 | (94.4%) | 19 | (95.0%) |
| | Missing | 5 | | 3 | |
| Change from Baseline | N | 18 | | 20 | |
| | Mean ± SD | 2.50 ± 5.43 | | −0.20 ± 6.30 | |
| | Median (P25, P75) | 3.50 | (−2.00, 7.00) | 0.00 | (−2.50, 4.50) |
| | Min, Max | −9.00, 10.00 | | −14.00, 10.00 | |
| | P-value (within-group) | 0.0673 | (T-Test) | 0.8887 | (T-Test) |

TABLE 12
Effects of the intake of soy kefir powder of the present invention on creatinine clearance

| | | Soy Kefir | | Placebo | |
|---|---|---|---|---|---|
| Creatinine Clearance (mL/min) | | (n = 23) | | (n = 23) | |
| Baseline | N | 18 | | 18 | |
| | Mean ± SD | 81.8 ± 20.1 | | 81.8 ± 20.1 | |
| | Median (P25, P75) | 80.0 | (65.8, 90.2) | 80.0 | (65.8, 90.2) |
| | Min, Max | 59.0, 128.1 | | 59.0, 128.1 | |
| | Missing | 23 | | 23 | |
| End of Treatment | N | 17 | | 17 | |
| | Mean ± SD | 79.4 ± 21.3 | | 80.8 ± 18.1 | |
| | Median (P25, P75) | 80.9 | (66.7, 83.6) | 81.2 | (67.0, 87.0) |
| | Min, Max | 54.2, 125.0 | | 59.2, 124.9 | |
| | Normal | 0 | (0.0%) | 1 | (100.0%) |
| | Missing | 23 | | 22 | |
| Change from Baseline | N | 17 | | 17 | |
| | Mean ± SD | −3.07 ± 4.18 | | −0.52 ± 4.96 | |
| | Median (P25, P75) | −4.40 | (−6.05, 0.90) | −0.10 | (−3.70, 1.30) |
| | Min, Max | −9.70, 3.60 | | −10.80, 9.10 | |
| | P-value (within-group) | 0.0079* | (T-Test) | 0.6725 | (T-Test) |

TABLE 13
Effects of the intake of soy kefir powder of the present invention on GGT

| | | Soy Kefir | | Placebo | |
|---|---|---|---|---|---|
| GGT (U/L) | | (n = 23) | | (n = 23) | |
| Baseline | N | 23 | | 23 | |
| | Mean ± SD | 33.1 ± 20.8 | | 33.1 ± 20.8 | |
| | Median (P25, P75) | 30.0 | (18.0, 35.0) | 30.0 | (18.0, 35.0) |
| | Min, Max | 13.0, 106.0 | | 13.0, 106.0 | |
| | Normal | 22 | (95.7%) | 22 | (95.7%) |
| | High (not clinically significant) | 1 | (4.3%) | 1 | (4.3%) |
| End of Treatment | N | 18 | | 20 | |
| | Mean ± SD | 37.2 ± 38.9 | | 32.6 ± 23.8 | |
| | Median (P25, P75) | 27.5 | (17.0, 37.0) | 26.5 | (18.0, 38.5) |
| | Min, Max | 11.0, 179.0 | | 8.0, 112.0 | |
| | Normal | 15 | (83.3%) | 19 | (95.0%) |
| | High (not clinically significant) | 3 | (16.7%) | 1 | (5.0%) |
| | Missing | 5 | | 3 | |
| Change from Baseline | N | 18 | | 20 | |
| | Mean ± SD | 1.61 ± 18.71 | | −1.45 ± 3.46 | |
| | Median (P25, P75) | −2.50 | (−6.00, 2.00) | −1.00 | (−3.00, 0.50) |
| | Min, Max | −14.00, 73.00 | | −10.00, 6.00 | |
| | P-value (within-group) | 0.1846 | (Signed Rank) | 0.0761 | (T-Test) |

TABLE 14
Effects of the intake of soy kefir powder of the present invention on urea nitrogen

| | | Soy Kefir | | Placebo | |
|---|---|---|---|---|---|
| Urea Nitrogen (mmol/L) | | (n = 23) | | (n = 23) | |
| Baseline | N | 23 | | 23 | |
| | Mean ± SD | 5.89 ± 1.21 | | 5.89 ± 1.21 | |
| | Median (P25, P75) | 6.10 | (5.20, 6.80) | 6.10 | (5.20, 6.80) |
| | Min, Max | 3.80, 7.80 | | 3.80, 7.80 | |
| | Normal | 21 | (91.3%) | 21 | (91.3%) |
| | High (not clinically significant) | 2 | (8.7%) | 2 | (8.7%) |
| End of Treatment | N | 18 | | 20 | |
| | Mean ± SD | 5.84 ± 1.24 | | 6.03 ± 1.45 | |
| | Median (P25, P75) | 6.00 | (4.70, 6.90) | 5.85 | (4.95, 7.25) |
| | Min, Max | 3.40, 7.90 | | 3.40, 8.40 | |
| | Normal | 17 | (94.4%) | 19 | (95.0%) |
| | High (not clinically significant) | 1 | (5.6%) | 1 | (5.0%) |
| | Missing | 5 | | 3 | |
| Change from Baseline | N | 18 | | 20 | |
| | Mean ± SD | 0.06 ± 0.84 | | 0.17 ± 1.03 | |
| | Median (P25, P75) | 0.10 | (−0.60, 0.60) | 0.00 | (−0.45, 0.95) |
| | Min, Max | −1.10, 1.70 | | −1.90, 2.60 | |
| | P-value (within-group) | 0.7812 | (T-Test) | 0.4825 | (T-Test) |

TABLE 15
Effect of storage on isoflavone content of the soy kefir powder of the present invention
Weeks of Storage

| Daidzin | 0* | 2 | 3 | 4 | 5 | 6 | 7 | 8 | 10 | 12 | 16 |
|---|---|---|---|---|---|---|---|---|---|---|---|
| 32 C. | 5.215 | 4.69 | 4.69 | 4.06 | 5.565 | 4.935 | | 4.62 | 5.50 | 5.495 | |
| 20 C. | 5.215 | | | | | | 4.48 | | | 4.95 | |
| 4 C. | | | | | 3.75 | | | 9.17 | | | 3.26 |
| Genistin | 0 | 2 | 4 | 5 | 6 | 7 | 8 | 9 | 10 | 12 | 16 |
| 32 C. | 17.22 | 16.8 | 16.49 | 10.22 | 15.05 | 15.82 | | 13.02 | 16.66 | 17.22 | |
| 20 C. | 17.22 | | | | | | 13.13 | | | 15.96 | |
| 4 C. | | | | | 14.60 | | | 19.46# | | | 11.62 |
| Total glycosides | 22.44 | 21.49 | 21.18 | 14.28 | 20.62 (32 C.) | 20.76 | 17.61 | 17.64 (32 C.) | | 22.69 (32 C.) | 14.88 (4 C.) |
| | | | | | 18.35 (4 C.) | | (20 C.) | 28.63 (4 C.) | | 20.81 (20 C.) | |
| Daidzein | 0 | 2 | 4 | 5 | 6 | 7 | 8 | 9 | 10 | 12 | 16 |
| 32 C. | 4.515 | 4.48 | 4.55 | 3.5 | 2.975 | 3.92 | | 2.975 | 3.29 | 3.22 | |
| 20 C. | 4.515 | | | | | | 2.555 | | | 3.01 | |
| 4 C. | | | | | 2.07 | | | 1.86 (4 C.) | | | 1.68 (4 C.) |
| Genistein | 0 | 2 | 4 | 5 | 6 | 7 | 8 | 9 | 10 | 12 | 16 |
| 32 C. | 8.225 | 8.12 | 7.63 | 6.195 | 7.98 | 8.54 | | 7.525 | 0.81 | 0.875 | |
| 20 C. | 8.225 | | | | | | 6.51 | | | 7.595 | |
| 4 C. | | | | | 5.81 | | | 5.85 | | | 4.73 |
| Total Aglycones | 12.74 | 12.6 | 12.18 | 9.695 | 10.96 (32 C.) | 12.46 | 9.065 | 10.5 (32 C.) | | 4.10 (32 C.) | |
| | | | | | 7.81 (4 C.) | | (20 C.) | 7.71 (4 C.) | | 10.61 (4 C.) | 6.41 (4 C.) |
| Total | 35.18 | 34.09 | 33.36 | 23.975 | 31.57 (32 C.) | 33.22 | 26.675 | 28.14 (32 C.) | 26.18 | 28.41 (20 C.) | |
| Isoflavones | | | | | 26.23 (4 C.) | (32 C.) | (20 C.) | 36.34 (4 C.) | (32 C.) | 26.79 (32 C.) | 21.29 (4 C.) |

*The Day 0 samples refer to samples that were analyzed approximately one month after manufacture and stored at room temperature. The 4° C. samples were stored at room temperature by KGK for one month and thus there is no Day 0 for these samples. Thus, the trendlines show different Day 0 time points for the 4° C. samples as opposed to the 32° C. and 20° samples. The Day 0 for the 20° C. and 32° C. samples actually refers to analysis of samples after approximately one month of storage at room temperature.

TABLE 16
Isoflavone content per 35 g soy kefir powder Stored at Different

| | | | | | | Estimated |
|---|---|---|---|---|---|---|
| | | | | | | level of |
| | Fermentation | Spray drying | | | | isoflavones - |
| | Dates | dates | Powder produced | Principal use | Anecdotal cases | November 2006 |
| 2004 | 10 batches from | From | Powder conserved in air | Clinical trial | Anecdotal studies on pain | High, over |
| | August 17 to | September 2 to | tight pouches.. | on blood | showed potency up to a year | 35 mg. |
| | August 31 | September 13. | Maintained out of cold | pressure | following fermentation | |
| | | | at room temperature. | | production. | |
| 2005 | 2 batches on | 13 Oct. | Not tested. Not | Pilot study on | No anecdotal studies on pain | |
| | October 10 and | 2005 | available. | pain | performed. Used in pilot | |
| | 11 | | | | study on pain relief described | |
| | | | | | in present patent application. | |
| 2005 | 2 batches on | 8 Nov. | Powder conserved in | Pilot study on | Tested several times for | High, over |
| | November 3 | 2005 | plastic bags | pain | anecdotal studies (Dr. | 35 mg. |
| | and 4 | | (maintained in cold but | | Garrel) from February 2006 | |
| | | | with interruptions) | | to August 2006. Good | |
| | | | | | results on pain relief. | |
| | | | | | Tested in May and June | |
| | | | | | 2006 on pain. Good results | |
| | | | | | on pain relief. | |
| 2006 | 3 batches on | From July 1^{st} to | Mix 63%/37% of | Stability tests | Tested formulated and mixed | High, over |
| | July 25, 26, 27 | August 2 | extruded powder and | | with the powder left in the | 35 mg. |
| | and 28 | (included) | powder stuck inside the | | spray dryer: | |
| | | | dryer. Mix formulated at | | | |
| | | | 70/30 with other | | | |
| | | | ingredients. | | | |

TABLE 17
Total Isoflavone and Individual Isoflavone Content (mg)
per 35 g soy kefir powder of the present invention stored at 4° C.

| Storage | | | Total | | | Total | Total | |
|---|---|---|---|---|---|---|---|---|
| Time | Daidzin | Genistin | Glycosides | Daidzein | Genistein | Aglycones | Isoflavones | % Aglycones |
| August | 16.73 | 23.49 | 40.22 | 2.94 | 6.55 | 9.49 | 49.7 | 19% |
| 2004 | | | | | | | | |
| November | 18.31 | 29.72 | 48.02 | 2.1 | 7.39 | 9.49 | 57.4 | 20% |
| 2005 | | | | | | | | |

TABLE 18
Sphingomyelin Content (ug/g) per 35 G soy kefir powder of the present
invention stored according to the invention at 30-32° C.

| | | | |
|---|---|---|---|
| Week 0 | Week 2 | Week 4 | Week 6 |

Storage Time (C16:16)

| | | | |
|---|---|---|---|
| 1030 | 27.8 | 22.6 | 23.8 |

Storage Time (C16:18)

| | | | |
|---|---|---|---|
| 32.4 | 5.8 | 22.8 | 11.8 |

Storage Time (C16:20)

| | | | |
|---|---|---|---|
| 19 | 10.4 | 5.8 | 6.6 |

Storage Time (C16:22)

| | | | |
|---|---|---|---|
| 7 | 2.6 | 3.8 | 4.4 |

(1 week = 1 month at 20° C.)

TABLE 19
Fatty Acid Composition of soy kefir powder of the
present invention at 4° C. at Different Storage Times
Stability Condition (4° C.)

| | Day | Week 4 | Week 8 |
|---|---|---|---|
| Fatty Acid | 0 | (Sep 5) | (Oct 11) |
| Myristic (14:0) | (P) | (W) | ND |
| Palmitic (16:0) | (P) | (P) | (P) |
| Linoleic (18:2n6) | (P) | (P) | (P) |
| Oleic (18:1) | (P) | (P) | (P) |
| Stearic (18:0) | (P) | (P) | (P) |
| α-Linolenic (18:3n3) | (W) | ND | (P) |
| Arachidic (20:0) | (P) | (W) | ND |
| Behenic (22:0) | (P) | (W) | ND |

TABLE 20
Fatty Acid Composition of soy kefir powder of the present invention at
30-32° C. at Different Storage Times
Stability Condition (30-32° C.)

| Fatty Acid | Day 0 | Week 4 | Week 6 | Week 7 | Week 8 |
|---|---|---|---|---|---|
| Myristic (14:0) | (P) | ND | ND | ND | ND |
| Palmitic (16:0) | (P) | (P) | (P) | (P) | (P) |
| Linoleic (18:2n6) | (P) | (P) | (P) | (P) | (P) |
| Oleic (18:1) | (P) | (P) | (P) | (P) | (P) |
| Stearic (18:0) | (P) | (P) | (P) | (P) | (P) |
| α-Linolenic | (W) | ND | (P) | (P) | (P) |
| (18:3n3) | | | | | |
| Arachidic (20:0) | (P) | (W) | ND | ND | ND |
| Behenic (22:0) | (P) | ND | ND | ND | ND |

P = Positive
W = Weak probability
ND = Not detectable

TABLE 21
Total Amino Acid Content per 35 g soy kefir powder of the present
invention Stored at 30-32° C.
Storage Time

| Week 0 | Week 2 | Week 4 | Week 6 | Week 7 | Week 8 | Week 9 |
|---|---|---|---|---|---|---|
| 44 626 736 | 66 671 064 | 78 997 602 | 70 860 824 | 71 280 038 | 67 686 742 | 80 082 801 |

TABLE 22
Total Amino Acid Content per 35 g soy kefir
powder of the present invention Stored at 4° C.
Storage Time

| Week 0 | Week 4 | Week 8 |
|---|---|---|
| 67 777 686 | 59 180 531 | 109 273 391 |

TABLE 23
Total Peptide Content per 35 g Soy Kef Powder of the present invention
Stored at 30-32° C.
Storage Time

| Week 0 | Week 2 | Week 4 | Week 6 | Week 7 | Week 8 | Week 9 |
|---|---|---|---|---|---|---|
| 322 580 253 | 546 790 417 | 872 192 380 | 515 203 126 | 609 488 550 | 907 975 594 | 710 408 459 |

TABLE 24
Total Peptide Content per 35 g soy kefir
powder of the present invention Stored at 4° C.
Storage Time

| Week 0 | Week 4 | Week 8 |
|---|---|---|
| 451 899 502 | 628 255 972 | 615 546 062 |

TABLE 25
Details of study procedures of Example 4

| | Screening | | Treatment Period I | | Washout Phase | | Treatment Period II | |
|---|---|---|---|---|---|---|---|---|
| | [Placebo Protein] | Baseline | Safety Visit | End-Tx | Safety Visit | End-Wash | Safety Visit | End-Tx |
| | Visit 1A-1O* | Visit 2 | Visit 3 | Visit 4 | Visit 5 | Visit 6 | Visit 7 | Visit 8 |
| | Wk −4 to 0 | Wk 0 | Wk 2 | Wk 4 | Wk 6 | Wk 8 | Wk 10 | Wk 12 |
| Informed Consent | X | | | | | | | |
| Inclusion/Exclusion Criteria | X | X | | | | | | |
| Medical History | X | | | | | | | |
| Physical Exam | X | | | X | | | | X |
| Urine Pregnancy Test | X | X | | | | X | | X |
| Randomization | | X | | | | | | |
| Study Treatment | | | | | | | | |
| Disperse | D | D | | | | D | | |
| Collect | | C | | C | | | | C |
| Office BP Measurement | X | X | X | X | X | X | X | X |
| ECG | | X | | X | | | | |
| Blood Sampling | | X | | X | | | | X |
| 24-hr ABPM | | X | | X | | | | X |
| Concomitant Medications | X | X | X | X | X | X | X | X |
| Adverse Events | | X | X | X | X | X | X | X |

*At Visit 1B the subject's Office BP will be recorded and subject qualification will be considered. The subject will qualify to immediately complete all procedures outlined for the Visit 2 (Baseline) if a) GDP is 140, 180 mmHg, and b) it is the Investigator's opinion that the subject's mean [symbol] SBP (ASPM) would be ≧135 mmHg. If the subject does not qulaify [symbol] [symbol] will attend Visit 1C in approximately 1 week, and Visit 1D approximately 1 week after that, if necessary, Subjects failing to qualify by Visit 1D will be considered as [symbol] failures and will be discontinued from further study participation. [symbol]
indicates data missing or illegible when filed

### REFERENCES

1. Zourari A, Anifantakis E M. Le kéfir: Caractères physicochimiques, microbiologiques et nutritionnels. Technologie de production. Une revue. Lait. 1988; 68:373-392.
2. Encyclopaedia of Food Science, Food Technology, and Nutrition. Macrae R, Robinson R K, Sadler M J, eds. Elsevier (UK), 1993, 1804-8.
3. Garrote G L, Abraham A G, De Antoni G L. Chemical and microbiological characterisation of kefir grains. J Dairy Res. 2001; 68:639-52.
4. Bottazzi V, Zacconi C, Sarra P G, Dallavalle P, Parisi M G. Kefir: Microbiologica, chimica e tecnologia. Industr Latte. 1994; 30:41-62.
5. Hallé C, Leroi F, Dousset X, Pidoux M. Les kéfirs: des associations bactéries lactiques—levures. In: Bactéries lactiques: Aspects fondamentaux et technologiques. Vol 2. Roissart H de, Luquet F M, eds. Uriage (France), 1994, 169-82.
6. Koroleva N S. Technology of kefir and kumys. IDF Bull. 1988; 227:96-100.
7. Molska I, Kocon J, Zmarlicki S. Electron microscopy studies on structures and microflora of kefir grains. Acta Alimentaria Polonica. 1980; 6:145-54.
8. Toba T, Arihara K., Adachi S. Distribution of microorganisms with particular reference to encapsulated bacteria in kefir grains. Int J Food microbiology. 1990; 10219-24.
9. Angulo L, Lopez E, Lema C. Microflora present in kefir grains of the Galician Region (North-West of Spain). J Dairy Res. 1993; 60:263-7.
10. Takizawa S, Kojima S, Tamura S, Fujinaga S, Benno Y, Nakase T. Lactobacillus kefirgranum sp. nov. and Lactobacillus parakefir sp. nov., Two New Species from Kefir Grains. Int J Syst Bacteriol. 1994; 44:435-9.
11. Loretana T, Mosterta J F, Viljoen B C. Microbial flora associated with South African household kefir. S Afr J. Sci. 2003; 99 No. ½.
12. Rosi J. Kefir micro-organisms: yeasts. Scienza e Tecnica Lattiero-Casearia. 1978; 29:59-67.
13. Komai M, Nanno M. Intestinal microflora and longevity. In: Functions of fermented milk. Nakazawa Y, Hosono A, eds. Elsevier Applied Science (London), 1992, 343.
14. Kroger M. Kefir. Cultured Dairy Prod J. 1993; 28:26-9.
15. Cevikbas A, Yemni E, Ezzedenn F W, Yardimici T. Antitumoural, antibacterial and antifungal activities of kefir and kefir grain. Phytother Res. 1994; 8:78-82.
16. Biffi A, Coradini D, Larsen R, Riva L, Di Fronzo G. Antiproliferative effect of fermented milk on the growth of a human breast cancer cell line. Nutr and Cancer. 1997; 28:93-99.
17. Furukawa N, Matsuoka A, Yamanaka Y. Effects of orally administered yogurt and kefir on tumor growth in mice. J Japan Soc Nutr Food Sci. 1990; 43:450-453.
18. Furukawa N, Matsuoka A, Takahashi T, Yamanaka Y. Effects of fermented milk on the delayed-type hypersensitivity response and survival day in mice bearing Meth-A. Anim Sci Technol. 1991; 62:579-85.
19. Zacconi C, Parisi M G, Sarra P G, Dallavalle P, Bottazzi V. Competitive exclusion of Salmonella kedougou in kefir fed chicks. Microbiol. Alim Nutr. 1995; 12:387-90.
20. Lu K-M. Methods for inhibiting cancer growth, reducing infection and promoting general health with a fermented soy extract. US Patent Publication No. 20020182274 filed Mar. 21, 2001.
21. Lu K-M. Methods for inhibiting cancer growth, reducing infection and promoting general health with a fermented soy extract. US Patent Publication No. 20030008023 filed Jul. 11, 2002.
23. International Dairy Federation: General standard of identity for fermented milks. 1992; 163, 4 p.
24. Mullally M M, Meisel H, FitzGerald R J. Identification of a novel angiotensin-I-converting enzyme inhibitory peptide corresponding to a tryptic fragment of bovine â-lactoglobulin. FEBS Lett. 1997; 402:99-101.
25. Okamoto A, Hanagata H, Matsumoto E, Kawamura Y, Koizumi Y, Yanagida F. Angiotensin I converting enzyme inhibitory activities of various fermented foods. Biosci Biotechnol Biochem. 1995; 59:1147-9.
26. Gobbetti M, Ferranti P, Smacchi E, Goffredi F, Addeo F. Production of angiotensin-I-converting enzyme inhibitory peptides in fermented milks started by Lactobacilus delbruekii subsp. bulgaricus SS1 and Lactococcus lactis subsp cremoris F T4. Appl Envir Microbiol. 2000; 66:3898-3904.
27. Kuwabara Y, Nagai, S, Yoshimitsu N, Nakagawa I, Watanabe Y, Tamai Y. Antihypertensive effect of the milk fermented by culturing with various lactic acid bacteria and a yeast. J Ferment Bioeng. 1995; 80; 294-5.
28. Nakamura Y, Yamamoto N, Saki K, Okubo A, Yamazaki S, Takano T. Purification and characterisation of angiotensin I converting enzyme inhibitors from sour milk. J Dairy Sci. 1995; 78:777-83.
29. Hernandez-Ledesma B, Amigo L, Ramos M, Recio I. Angiotensin converting enzyme inhibitory activity in commercial fermented products. Formation of peptides under simulated gastrointestinal digestion. J Agric Food Chem. 2004; 52:1504-10.
30. Chang, B W, Chen R L, Huang I J, Chang H C. Assays for angiotensin converting enzyme inhibitory activity. Ana. Biochem. 2001; 291:84-8.
31. Church F C, Swaisgood H E, Porter D H, Catignani G L. Spectrophotometric assay using ophthaldialdehyde for determination of proteolysis in milk and isolated milk proteins. J Dairy Sci. 1983; 66:1219-1227.
32. Hernandez-Ledesma, B, Amigo L, Ramos M, Recio I. Angiotensin converting enzyme inhibitory activity in commercial fermented products. Formation of peptides under simulated gastrointestinal digestion. J Agric Food Chem. 2004; 52:1504-10.
33. Shin Z, Yu R, Park S-A, Chung D K, Ahn C W, Nam H S, Kim K S, Lee H J. 2001. His-His-Leu, an angiotensin I converting enzyme inhibitory peptide derived from korean soybean paste, exerts antihypertensive activity in vivo. J Agric Food Chem. 2001; 49:3004-9.
34. Wu J, Ding X. Hypotensive and Physiological Effect of Angiotensin Converting Enzyme Inhibitory Peptides Derived from Soy Protein on Spontaneously Hypertensive Rats. J Agric Food Chem. 2001; 49:501-6.
35. Shin Z I, Ahn C W, Nam H S, Lee H J, Lee H J, Moon T H. Fractionation of angiotensin converting enzyme inhibitory peptide from soybean paste. Korean J Food Sci Technol. 1995; 27:230-4.
36. Weir M R, Dzau V J. The renin-angiotensin-aldosterone system: a specific target for hypertension management. Am J. Hypertens. 1999; 12:205 S-213S.
37. Brasier A R, Recinos A, Eledrisi M S. Vascular inflammation and the renin-angiotensin system. Arterioscler Thromb Vasc Biol. 2002; 22:1257-66.
38. Walker W G, Whelton P K, Saito H, Russel R P, Hermann J. Relation between blood pressure and renin, renin substrate, AII, aldosterone and urinary sodium and potassium in 574 ambulatory subjects. Hypertension. 1979; 1:287-91.
39. Gabay C, Kushner I. Acute-phase proteins and other systemic responses to inflammation. N Engl J. Med. 1999; 340:448-54.
40. Sesso H D, Buring J E, Rifai N, Blake G J, Gaziano J M, Ridker P M. C-reactive protein and the risk of developing hypertension. JAMA. 2003; 290:2945-51.
41. Raji A. Seely E W. Bekins S A. Williams G H. Simonson D C. 2003. Rosiglitazone improves insulin sensitivity and lowers blood pressure in hypertensive patients. Diabetes Care. 26:172-8.
42. Pradhan A D, Manson J E, Rossouw J E, Siscovick D S, Mouton C P, Rifai N, Wallace R B, Jackson R D, Pettinger M B, Ridker P M. Inflammatory biomarkers, hormone replacement therapy, and incident coronary heart disease: prospective analysis from the Women's Health Initiative observational study. JAMA 2002; 288:980-7.
43. Adlercreutz H, Mazur W. Phyto-oestrogens and Western diseases. Ann Med. 1997; 29:95-120.
44. Fukutake M, Takahashi M, Ishida K, Kawamura H, Sugimura T, Wakabayashi K. Quantification of genistein and genistin in soybeans and soybean products. Food Chem. Toxicol. 1996; 34:457-61.
45. Martin D S, Breitkopf N P, Eyster K M, Williams J L. Dietary soy exerts an antihypertensive effect in spontaneously hypertensive female rats. Am J Physiol Regul Integr Comp Physiol. 2001; 281:R553-60.
46. Martinez R M, Gimnénez I, Lou J M, Mayoral J A, Alda J O. Soy isoflavonoids exhibit in vitro biological activities of loop diuretics. Am J Clin Nutr. 1998; 68:1354s-1357s.
47. Nevala R, Korpela R, Vapaatalo H. Plant derived estrogens relax rat mesenteric artery in vitro. Life Sci. 1998; 63:95-100.
48. Gimenez I, Lou M, Vargas F, Alvarez-Guerra M, Mayoral J A, Martinez R M, Garay R P, Alda J O. Renal and vascular action of equol in the rat. J Hypertens 1997; 15:1303-8.
49. Mishra S K, Abbot S E, Choudhury Z, Cheng M, Khatab N, Maycock N J, Zavery A, Aaronson P I. Endothelium-dependent relaxation of rat aorta and main pulmonary artery by the phytoestrogens genistein and daidzein. Cardiovasc Res. 2000; 46:539-46.
50. Duarte J, Perez-Palencia R, Vargas F, Ocete M A, Perez-Vizcaino F, Zarzuelo A, Tamargo J. Antihypertensive effects of the flavonoid quercetin in spontaneously hypertensive rats. Br J. Pharmacol. 2001; 133:117-24.
51. Sadowska-Krowicka H, Mannick E E, Oliver P D, Sandoval M, Zhang X J, Eloby-Childess S, Clark D A, Miller M J. Genistein and gut inflammation: role of nitric oxide. Proc Soc Exp Biol Medicine. 1998; 217:351-7.
52. Kong L Y, Lai C, Wilson B C, Simpson J N, Hong J S. Protein tyrosine kinase inhibitors decrease lipopolysaccharide-induced proinflammatory cytokine production in mixed glia, microglia-enriched or astrocyte-enriched cultures. Neurochem Int. 1997; 30:491-7.
53. Deodato B, Altavilla D, Giovanni S. Cardioprotection by the phytoestrogen genistein in experimental myocardial ischemia-reperfusion injury. Br J. Pharmacol. 1999; 128:1683-90.
54. Regal J F, Fraser D G, Weeks C E, Greenberg N A. Dietary Phytoestrogens Have Anti-Inflammatory Activity in a Guinea Pig Model of Asthma. Proc Soc Exp Biol Med. 2000; 223:372-8.
55. Guven A, Guven A, Gulmez M. The effect of kefir on the activities of GSH-Px, GST, CAT, GSH and LPO levels in carbon tetrachloride-induced mice tissues. J Vet Med B Infect Dis Vet Public Health. 2003; 50:412-6.
56. Kubow S, Foutouhinia M. Kefir as a potent antioxidant composition. U.S. Patent Ser. No. 60/213,268 filed Jun. 22, 2000. International Patent Application No. PCT/CA01/00899 filed Jun. 18, 2001.
57. Alm L. Survival rate of Salmonella and Shigella in fermented milk products with and without added human gastric juices: An in vitro study. Prog Food Nutr Sci. 1983; 7:19-26.
58. Korneva V V, Nabukhotnyi T K, Cherevko S A, Kravets A A, Sidorchuk I I. Use of propiono-acidophylus milk in the complex treatment of intestinal disbacteriosis in infants with taphylococcal infections and sepsis. Vopr Pitan. 1979; 5:37-40.
59. Morgan S M, Hickey R, Ross R P, Hill C. Efficient method for the detection of microbially-produced antibacterial substances from food systems. J Appl Microbiol. 2000; 89:56-62.
60. Batinkov E L. Use of milk and kefir in peptic ulcer of the stomach and duodenum. Vopr Pitan. 1971; 30:89-91.
61. Murofushi M, Shiomi M, Aibara K. Effect of orally administered polysaccharide from kefir grain on delayed-type hypersensitivity and tumor growth in mice. Jpn J Med Sci Biol. 1983; 36:49-53.
62. Osada K, Nagira K, Teruya K, Tachibana H, Shirahata S, Murakami H. Enhancement of interferon-b production with sphingomyelin from fermented milk. Biother. 1994; 7:115-123.
63. Thoreux K, Schmucker D L. Kefir milk enhances intestinal immunity in young but not old rats. J. Nutr. 2001; 131:807-12.
64. Kubow S, Chan H M, Chen C, Fotouhinia M. Kefir extract as an anti-cancer agent. U.S. Patent Ser. No. 60/211,804 filed Jun. 16, 2000. International Patent Application No. PCT/CA01/00896 filed Jun. 15, 2001.
65. Liu J R, Wang S Y, Lin Y Y, Lin C W. Antitumor activity of milk kefir and soy milk kefir in tumor-bearing mice. Nutr Cancer. 2002; 44:182-187.
66. Murofushi M, Mizuguchi J, Aibara K, Matuhasi T. Immunopotentiative effect of polysaccharide from kefir grain, KGF-C, administered orally in mice. Immunopharmacology 1986; 121:29-35.
67. Kubo M, Odani T, Nakamura S, Tokumaru S, Matsuda H. Pharmacological study on Kefir—a fermented milk product in Caucasus. I. On antitumor activity. Yakugaku Zasshi. 1992; 112:489-95.
68. Shiomi M, Sasaki K, Murofushi M, Aibara K. Antitumor activity in mice of orally administered polysaccharide from Kefir grain. Jpn J Med Sci Biol. 1982; 35:75-80.
69. Shiomi M, Aibara K, Murofushi M. Effect of orally administered polysaccharide from kefir grain on delayed-type hypersensitivity and tumor growth in mice. Jpn J Med Sci Biol. 1983; 36:49-53.
70. Alm L. The effect of fermentation on the biological value of milk proteins evaluated using rats. A study on Swedish fermented milk products. J Sci Food Agric. 1981; 32:1247-53.
71. Astwood J D, Leach J N, Fuchs R L. Stability of food allergens to digestion in vitro. Nat. Biotechnol. 1996; 14:1269-73.
72. Kiers J L, Van Laeken A E, Rombouts F M, Nout M J. In vitro digestibility of bacillus fermented soya bean. Int J Food Microbiol. 2000; 60:163-9.
73. Silk D B, Hegarty J E, Fairvlough P D, Clark M L. Characterization and nutritional significance of peptide transport in man. Ann Nutr Metab. 1982; 26:337-52.
74. Roberts P R, Burney J D, Black K W, Zaloga G P. Effect of chain length on absorption of biologically active peptides from the gastointestinal tract. Digestion. 1999; 26:222-227.
75. Masuda O, Nakamura Y, Takano T. Antihypertensive peptides are present in aorta after oral administration of sour milk containing these peptides to spontaneously hypertensive rats. J. Nutr. 1996; 126:3063-8.
76. Morton M S, Wilcox G, Wahlqvist M L, Griffiths K. Determination of lignans and isoflavonoids in human female plasma following dietary supplementation. J. Endocrinol. 1994; 142:251-9.
77. Manach C, Scalbert A, Morand M, Rémésy C, Jiménez L. Polyphenols: food sources and bioavailability. Amer J Clin Nutr. 2004; 79:727-47.
78. Kinoshita E, Yamakoshi J, Kikuchi M. Purification and Identification of an Angiotensin I-Converting Enzyme Inhibitor from Soy Sauce. Biosci Biotechnol Biochem. 1993; 57:1107-10.
79. Akiko O, Hiroshi H, Eiko, M. Antihypertensive substances in viscous material of fermented soybean (NATTO). In: Food Hydrocolloids: Structures, Properties, and Functions. Nishinar K, Doi E, eds. Plenum Press (New York), 1994, 497-502.
80. Fitzgerald R J, Murray B A, Walsh D J. Hypotensive peptides from milk proteins. J. Nutr. 2004; 134: 980S-988S.
81. Anon: Foods interacting with MAOI inhibitors. Med Lett Drug Ther. 1989; 31:11-12.
82. Nakamura Y, Yamamoto N, Sakai K, Takano T. Antihypertensive effect of sour milk and peptides isolated from it that are inhibitors to angiotensin I-converting enzyme. J Dairy Sci. 1995; 78:1253-7.
83. Messina M, Flickinger B. Hypothesized anticancer effects of soy: evidence points toward isoflavones as the primary anticarcinogens. Pharm Biol. 2002; 40:6 S-23S.
84. Nagata C, Takatsuka N, Kurisu Y, Shimizu H. Decreased serum total cholesterol concentration is associated with high intake of soy products in Japanese men and women. J. Nutr. 1998; 128:209-13.
85. Zhang X, Shu X O, Gao Y T, Yang G, Li Q, Li H, Jin F, Zheng W. Soy food consumption is associated with lower risk of coronary heart disease in chinese women. J. Nutr. 2003; 133:2874-8.
86. Horiuchi T, Onouchi T, Takahashi M, Ito H, Orimo H. Effect of soy protein on bone metabolism in postmenopausal Japanese women. Osteoporos Int. 2000; 11:721-4.
87. Messina M. Isoflavone intakes by Japanese were overestimated. Am J Clin Nutr. 1995; 62:645.
88. Adlercreutz H, Honjo H, Higashi A, Fotsis T, Hamalainen E, Hasegawa T, Okada H. Urinary excretion of lignans and isoflavonoid phytoestrogens in Japanese men and women consuming a traditional Japanese diet. Am J Clin Nutr. 1991; 54:1093-100.
89. Kimira M, Arai Y, Shimoi K, Watanabe S. Japanese intake of flavonoids and isoflavonoids from foods. J. Epidemiol. 1998; 8:168-75.
90. Setchell K D R, Zimmer-Nechemias L, Cai J, Heubi J E. Exposure of infants to phyto-oestrogens from soy-based infant formula. Lancet. 1997; 350:23-7.
91. Svanberg U, Sandberg A S. Improved iron availability in weaning foods using germination and fermentation. In: Nutrient Availability: Chemical and Biological Aspects. Southgate D A T, Johnson I T, Fenwick G R, eds. Cambridge University Press (UK), 1989, 179-81.
92. Rivas M, Garay R P, Escanero J F, Cia Jr P, Cia P, Alda J O. Soy Milk Lowers Blood Pressure in Men and Women with Mild to Moderate Essential Hypertension. J. Nutr. 2002; 132:1900-2.
93. Seppo L, Jauhiainen T, Poussa T, Korpela R. A fermented milk high in bioactive peptides has a blood pressure-lowering effect in hypertensive subjects. Am J Clin Nutr. 2003; 77:326-30.
94. Hata Y, Yamamoto M, Ohni M, Nakajima K, Nakamura Y, Takano T. A placebo controlled study of the effect of sour milk on blood pressure in hypertensive subjects. Am J Clin Nutr. 1996; 64:767-71.
95. Inoue K, Shirai T, Ochiai H, Kasao M, Hayakawa K, Kimura M, Sansawa H. Blood-pressure-lowering effect of a novel fermented milk containing gamma-aminobutyric acid (GABA) in mild hypertensives. Eur J Clin Nutr. 2003:57:490-5.
96. Kurmann J A, Rasic J L, Kroger M. Encyclopedia of fermented fresh milk products: an international inventory of fermented milk, cream, buttermilk, whey and related products. Van Nostrand Reinhold Company (New York), 1992, 368 pp.
97. Livingston M G, Livingston H M. Monoamine oxidase inhibitors. An update on drug interactions. Drug Safety. 1996; 14:219-27.
98. Divi R L, Chang H C, Doerge D R. Anti-thyroid isoflavones from soybean: isolation, characterization, and mechanisms of action. Biochem Pharmacol. 1997; 54:1087-96.
99. A M Cuevas, V L Irribarra, O A Castillo, M D Y and A M Germain. Isolated soy protein improves endothelial function in postmenopausal hypercholesterolemic women. Eur. J. Clin. Nutr. 2003, 57: 889-894
100. Hodgson J M, Puddey I B, Beilin L J, Mori T A, Burke V, Croft K D, Rogers P B. Effects of isoflavonoids on blood pressure in subjects with high-normal ambulatory blood pressure levels: a randomized controlled trial. Am J. Hypertens. 1999 January; 12 (1 Pt 1):47-53.
101. Squadrito F, Altavilla D, Morabito N, et al. The effect of the phytoestrogen genistein on plasma nitric oxide concentrations, endothelin-1 levels and endothelium dependent vasodilation in postmenopausal women. Atherosclerosis 2002; 163:339-47.
102. Simons L A, von Konigsmark M, Simons J, Celermajer D S. Phytoestrogens do not influence lipoprotein levels or endothelial function in healthy, postmenopausal women. Am J Cardiol 2000; 85:1297-301.
103. Atkinson C. Oosthuizen W. Scollen S. Loktionov A. Day N E. Bingham S A. Modest protective effects of isoflavones from a red clover-derived dietary supplement on cardiovascular disease risk factors in perimenopausal women, and evidence of an interaction with ApoE genotype in 49-65 year-old women. Journal of Nutrition. 134:1759-64, 2004.
104. S. Kreijkamp-Kaspers, L. Kok, M. L Bots, D. E. Grobbee, J. W. Lampe and Y. T. van der Schouw. Randomized controlled trial of the effects of soy protein containing isoflavones on vascular function in postmenopausal women. American Journal of Clinical Nutrition, Vol. 81, No. 1, 189-195, 2005.
105. Meyer B J. Larkin T A. Owen A J. Astheimer L B. Tapsell L C. Howe P R. Limited lipid-lowering effects of regular consumption of whole soybean foods. Annals of Nutrition & Metabolism. 48:67-78, 2004.
106. Allison D B. Gadbury G. Schwartz L G. Murugesan R. Kraker J L. Heshka S. Fontaine K R. Heymsfield S B. A novel soy-based meal replacement formula for weight loss among obese individuals: a randomized controlled clinical trial. European Journal of Clinical Nutrition. 57:514-22, 2003.
107. Washburn S, Burke G L, Morgan T, Anthony M. Effect of soy protein supplementation on serum lipoproteins, blood pressure, and menopausal symptoms in perimenopausal women. Menopause. 1999 Spring; 6 (1):7-13.
108. D. J. A. Jenkins, C. W. C. Kendall, C.-J. C. Jackson, P. W. Connelly, T. Parker, D. Faulkner, E. Vidgen, S. C. Cunnane, L. A. Leiter and R. G. Josse. Effects of high- and low-isoflavone soyfoods on blood lipids, oxidized LDL, homocysteine, and blood pressure in hyperlipidemic men and women. Am. J. Clinical Nutr., Vol. 76, No. 2, 365-372, August 2002.
109. Nakamura M. Aoki N. Yamada T. Kubo N. Feasibility and effect on blood pressure of 6-week trial of low sodium soy sauce and miso (fermented soybean paste). Circulation Journal. 67 (6):530-4, 2003
110. Hasler C M. The cardiovascular effects of soy products. Journal of Cardiovascular Nursing. 16 (4):50-63; 2002.
111. Sekiya, S., Kobayashi, Y., Kita, E., Imamura, Y. & Toyama, S. (1992) Antihypertensive effects of tryptic hydrolysate of casein on normotensive and hypertensive volunteers (in Japanese). J. Jap. Soc. Nutr. Food Sci. 45:513-517.
112. Hata, Y., Yamamoto, M., Ohni, M., Nakajima, K., Nakamura, Y. & Takano, T. (1996) A placebo-controlled study of the effect of sour milk on blood pressure in hypertensive subjects. Amer. J. Clin. Nutr. 64:767-771.
113. Seppo, L., Kerojoki, O., Suomalainen, T. & Korpela, R. (2002) The effect of a Lactobacillus helveticus L BK-16H fermented milk on hypertension—a pilot study on humans. Milchwissen 57:124-127.
114. Seppo, L., Jauhiainen, T., Poussa, T. & Korpela, R. (2003) A fermented milk high in bioactive peptides has a blood pressure-lowering effect in hypertensive subjects. Am. J. Clin. Nutr. 77:326-330.
115. Nimmagudda, R. (2002) New bioactive peptides for the nutritional industry. Oral presentation at: SupplySide West Trade Show and Conference 2002 Las Vegas, Nev. December 4th to 6th.
116. Pins, J. J. & Keenan, J. M. (2002) The antihypertensive effects of a hydrolysed whey protein isolate supplement (BioZate® 1). Cardiovasc. Drugs Ther. 16:68.
117. Inoue K, Shirai T, Ochiai H, Kasao M, Hayakawa K, Kimura M, Sansawa H. Blood-pressure-lowering effect of a novel fermented milk containing gamma-aminobutyric acid (GABA) in mild hypertensives. Eur J Clin Nutr. 2003 March; 57 (3):490-5.
118. Teede H J, Dalais F S, Kotsopoulos D, Liang Y L, Davis S, McGrath B P Dietary Soy Has Both Beneficial and Potentially Adverse Cardiovascular Effects: A Placebo-Controlled Study in Men and Postmenopausal Women. The Journal of Clinical Endocrinology & Metabolism 86, 3053-3060.
119. Rivas M, Garay R P, Escanero J F, Cia P Jr, Cia P, Alda J O. Soy milk lowers blood pressure in men and women with mild to moderate essential hypertension. J. Nutr. 2002 July; 132 (7):1900-2.
120. Sagara M, Kanda T, N Jelekera M, Teramoto T, Armitage L, Birt N, Birt C, Yamori Y. Effects of Dietary Intake of Soy Protein and Isoflavones on Cardiovascular Disease Risk Factors in High Risk, Middle-Aged Men in Scotland. J Am Coll Nutr. 2004 February; 23 (1):85-91.

1. A soy kefir powder obtained by fermenting soymilk with active kefir grains from the Moscow kefir strain, characterized in that it comprises a total isoflavone composition of approximately 0.1-0.4%.

2. The soy kefir powder of claim 1, wherein said powder having at least one biological activity selected from the group consisting of: pain relief, blood pressure reduction and inflammation reduction.

3. The soy kefir powder of claim 1, wherein the isoflavones are isoflavone gycosides or aglycones.

4. The soy kefir powder of claim 3, wherein the aglycones have a composition of approximately 0.01-0.03%.

5. The soy kefir powder of claim 3, wherein the aglycones are selected from the group consisting of genistein, daidzein and glycetein.

6. The soy kefir powder according to claim 1, wherein it further comprises at least one of the following:
a protein composition of approximately of 25-45%;
a carbohydrate composition of approximately of 25-45%;
a fat composition of approximately of 25-45%; and
an ash composition of approximately of 9-15%.

7. The soy kefir powder according to claim 6, wherein it further comprises at least one of the following:
a saturated fatty acids composition of approximately 2-3.5%;
a monounsaturated fatty acids composition of approximately 3.5-5.5%; and
an N-6 polyunasturated fatty acids composition of approximately 10-13%.

8. A method for preparing soy kefir powder having at least one biological activity selected from the group consisting of: pain relief, blood pressure reduction and inflammation reduction, said method comprising the steps of:
a—fermenting soymilk with active Moscow kefir grains under suitable fermentation conditions to obtain a fermentation culture, said soymilk being in a ratio ranging between 20/1 to 100/1 (volume/weight) of soymilk to active Moscow kefir grain;
b—separating the active Moscow kefir grains from the fermentation culture obtained in step a) to obtain a fermentation liquid; and
c—spray drying the fermentation liquid obtained in step b) to form a soy kefir powder.

9. The method of claim 8, wherein in step a) the soymilk is fermented with the active Moscow kefir grains during approximately 10 to 24 hours.

10. The method of claim 9, wherein in step a) the soymilk is fermented during approximately 16 to 24 hours.

11. The method of claim 10, wherein in step a) the soymilk is fermented during approximately 16 hours.

12. The method of claim 8, wherein in step a) the soymilk is fermented at a temperature ranging between 19 and 27° C.

13. The method of claim 8, wherein in step b) the active Moscow kefir grains are separated by coarse sieving or by draining the fermentation liquid.

14. The method of claim 8, wherein the fermentation liquid obtained in step b) is refrigerated.

15. The method of claim 14, wherein the fermentation liquid obtained in step b) is refrigerated for a duration of approximately 1 to 5 days at a temperature ranging approximately between 2 to 8° C.

16. The method of claim 8, wherein the fermentation liquid is spray-dried at a temperature of 65° C.±13° C.

17. The method of claim 8, wherein in step a) the pH of the fermentation culture is between 4.6 and 5.0.

18. The method of claim 17, wherein in step a) the pH of the fermentation culture is 4.8.

19. Soy kefir powder obtained by the method of claim 8.

20. The soy kefir powder according to claim 19, wherein that it comprises a total isoflavone composition of approximately 0.1-0.4%.

21. The soy kefir powder of claim 20, wherein the isoflavones are isoflavone gycosides or aglycones.

22. The soy kefir powder of claim 21, wherein the aglycones have a composition of approximately 0.01-0.03%.

23. The soy kefir powder of claim 22, wherein the aglycones are selected from the group consisting of genistein, daidzein and glycetein.

24. The soy kefir powder according to claim 19, wherein it further comprises at least one of the following:
a protein composition of approximately of 25-45%;
a carbohydrate composition of approximately of 25-45%;
a fat composition of approximately of 25-45%; and
an ash composition of approximately of 9-15%.

25. The soy kefir powder according to claim 24, wherein it further comprises at least one of the following:
a saturated fatty acids composition of approximately 2-3.5%;
a monounsaturated fatty acids composition of approximately 3.5-5.5%; and
an N-6 polyunasturated fatty acids composition of approximately 10-13%.

26. Use of the soy kefir powder according to claim 1, for pain relief in a subject in need thereof.

27. Use of the soy kefir powder according to claim 1, for blood pressure reduction in a subject in need thereof.

28. Use of the soy kefir powder according to claim 1, for inflammation reduction in a subject in need thereof.

29. A method for pain relief in a subject in need thereof, comprising the step of administering to said subject an effective amount of a soy kefir powder as defined in claim 1.

30. A method for blood pressure reduction in a subject in need thereof, comprising the step of administering to said subject an effective amount of a soy kefir powder as defined in claim 1.

31. A method for inflammation reduction in a subject in need thereof, comprising the step of administering to said subject an effective amount of a soy kefir powder as defined in claim 1.
